(19) United States
(12) Patent Application Publication
(10) Pub. No.: US 2004/0248986 A1
(43) Pub. Date: Dec. 9, 2004

(54) Use of n,n-diethyl-2-{-4-(phenylmethyl) - phenoxy} ethanamine monohydrochloride (dppe) in cancer therapy

(76) Inventor: Mark Vincent, London (CA)

Correspondence Address: ROTHWELL, FIGG, ERNST & MANBECK, P.C., 1425 K STREET, N.W., SUITE 800, WASHINGTON, DC, 20005, US

(21) Appl. No.: 10/494,284

(22) PCT Filed: Nov. 1, 2002
(86) PCT No.: PCT/CA02/01651

(30) Foreign Application Priority Data
Nov. 1, 2001 (CA) 2,360,849

(51) Int. Cl. A61K 31/137

(52) U.S. Cl. 514/649

(57) ABSTRACT

The present invention provides for the use of N,N-diethyl-2-[-4-(phenylmethyl)-phenoxy]ethanamine monohydrochloride (DPPE) in cancer therapy. DPPE is used in the treatment of patients having, or suspected of having, an aggressive cancer. The present invention further provides for the use of DPPE in the treatment of a patient suspected of having an existing cancer, wherein the use follows a surgery for treatment of a primary cancer that is estrogen-receptor negative. Also provided are pharmaceutical compositions comprising DPPE for use in the treatment of patients having, or suspected of having, an aggressive cancer and pharmaceutical kits comprising such compositions.

### FIELD OF THE INVENTION

The present invention pertains to the use of N,N-diethyl-2-[-4-(phenylmethyl)-phenoxy]ethanamine monohydrochloride (DPPE), commonly known as Tesmilifene, in the treatment of cancer.

### BACKGROUND

Primary treatment for many cancers is some form of surgery to remove the cancerous tissue. Following primary therapy, patients at risk of relapsing often undergo adjuvant therapy, which is initiated soon after primary therapy in order to delay recurrence and/or to prolong survival. One kind of adjuvant systemic therapy is adjuvant chemotherapy, which involves administration of one or more chemotherapeutic agents.

The use of N,N-dialkyl-2-[(4-phenylmethyl)-phenoxy]ethanamine and N-morpholino-2-[(4-phenylmethyl)-phenoxy]ethanamine compounds and their salts, as anti-cancer agents, has been previously described. Compositions, including mixtures of these ethanamine compounds with therapeutically active anti-cancer compounds, such as doxorubicin, have been found particularly beneficial and have been previously described for use to treat breast and colon cancer.

N,N-diethyl-2-[-4-(phenylmethyl)-phenoxy]ethanamine monohydrochloride (DPPE) has been shown to inhibit the in vitro growth of MCF-7 breast cancer cells that are estrogen-receptor negative (ER−)/AEBS+, or ER+/AEBS+ (U.S. Pat. No. 4,803,227).

DPPE has also been shown to inhibit normal cell proliferation while promoting malignant cell proliferation in vivo in an animal model. DPPE is a potent antagonist selective for intracellular histamine receptors when administered in amounts sufficient to inhibit the binding of intracellular histamine to the receptors in normal and malignant cells. The same study indicated that DPPE can act synergistically with doxorubicin (Adriamycin™) in tumour-bearing animals treated concurrently with DPPE (International Patent Application WO92/11035; U.S. Pat. No. 5,859,065). It has been postulated that this effect was achieved through the use of DPPE at doses which inhibit the growth of normal cells, but which promote the growth of tumour cells, thus rendering the latter more susceptible to the cytotoxic effects of chemotherapeutic agents (U.S. Pat. No. 5,859,065).

More recently, based on competition assays, compounds such as N,N-diethyl-2-[4-(4′-fluorophenone)-phenoxy]ethanamine (DFPE) have been shown to act in a similar manner to DPPE but with greater potency. These studies suggest that DFPE also acts, at the appropriate amounts, to inhibit normal cell proliferation and promote malignant cell proliferation. It has been postulated that such compounds can be used to enhance the therapeutic index of conventional chemotherapy drugs (U.S. Pat. No. 6,284,799).

Rapidly growing, aggressive or metastatic cancers are particularly difficult to treat and patients with this type of cancer have significantly reduced survival rates. Typically, combinations of chemotherapeutic agents are used in the treatment of such patients in order to slow the growth of the cancer.

It has recently been demonstrated that DPPE is useful in enhancing the effect of chemotherapeutic agents in the treatment of hormone-unresponsive metastatic prostate cancer. An initial intravenous infusion of DPPE over an approximately one hour period prior to cyclophosphamide treatment was shown to potentiate the anti-cancer activity and ameliorate the toxicity associated with using cyclophosphamide, or other chemotherapeutic agents which are normally inactive against this type of cancer (U.S. Pat. No. 5,863,912).

Despite encouraging results in small scale clinical studies trials set up to test the effect of DPPE in combination with a second chemotherapeutic on various cancers (U.S. Pat. No. 5,859,065), a recent phase III clinical trial for breast cancer did not reveal a significant synergistic effect or potentiation of doxorubicin during, or shortly after, the treatment period. A need still exists, therefore, for an effective treatment of rapidly growing, aggressive or metastatic cancers.

This background information is provided for the purpose of making known information believed by the applicant to be of possible relevance to the present invention. No admission is necessarily intended, nor should be construed, that any of the preceding information constitutes prior art against the present invention. Publications referred to throughout the specification are hereby incorporated by reference in their entireties in this application.

### SUMMARY OF THE INVENTION

An object of the present invention is to provide a use of N,N-diethy-2-[4-(phenylmethyl)-phenoxy]ethanamine monohydrochloride (DPPE) in cancer therapy. In accordance with an aspect of the present invention, there is provided a use of DPPE in the treatment of a cancer patient having, or suspected of having, an aggressive cancer and thereby extending the survival of the patient.

In accordance with another aspect of the invention, there is provided a use of DPPE in the treatment of a patient suspected of having an existing cancer and thereby extending the survival of the patient, wherein the use follows a surgery for treatment of a primary cancer that is an estrogen-receptor negative cancer.

In accordance with another aspect of the invention, there is provided a use of DPPE to manufacture a medicament for the treatment of a patient having, or suspected of having, an aggressive cancer and thereby extend the survival of the patient.

In accordance with another aspect of the invention, there is provided a use of DPPE to manufacture a medicament for the treatment of a cancer patient having an existing cancer and thereby extend the survival of the patient, wherein the use follows a surgery for treatment of a primary cancer that is an estrogen-receptor negative cancer.

### BRIEF DESCRIPTION OF THE FIGURES

FIG. 1 provides a graphical representation of survival time of ER negative patients receiving DPPE and doxorubicin (DPPE/DOX) or doxorubicin alone (DOX).

FIG. 2 provides a graphical representation of survival time of ER positive patients receiving DPPE and doxorubicin (DPPE/DOX) or doxorubicin alone (DOX).

FIG. 3 provides a graphical representation of survival time of patients receiving doxorubicin alone, with a comparison of ER negative and ER positive patients.

FIG. 4 provides a graphical representation of survival time of patients receiving DPPE and doxorubicin, with a comparison of ER negative and ER positive patients.

FIG. 5 provides a graphical representation of survival time of patients having a duration of ≦6 months, receiving DPPE and doxorubicin (DPPE/DOX) or doxorubicin alone (DOX).

FIG. 6 provides a graphical representation of survival time of patients having a duration of >6 months to ≦36 months, receiving DPPE and doxorubicin (DPPE/DOX) or doxorubicin alone (DOX).

FIG. 7 provides a graphical representation of survival time of patients having a duration >36 months, receiving DPPE and doxorubicin (DPPE/DOX) or doxorubicin alone (DOX).

FIG. 8 provides a graphical representation of survival time of ER positive patients having a duration ≦36 months, receiving DPPE and doxorubicin (DPPE/DOX) or doxorubicin alone (DOX).

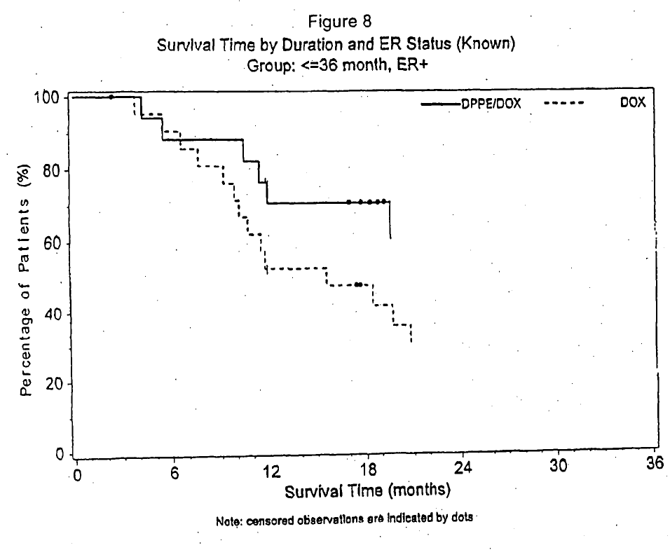

FIG. 9 provides a graphical representation of survival time of ER negative patients having a duration ≦36 months, receiving DPPE and doxorubicin (DPPE/DOX) or doxorubicin alone (DOX).

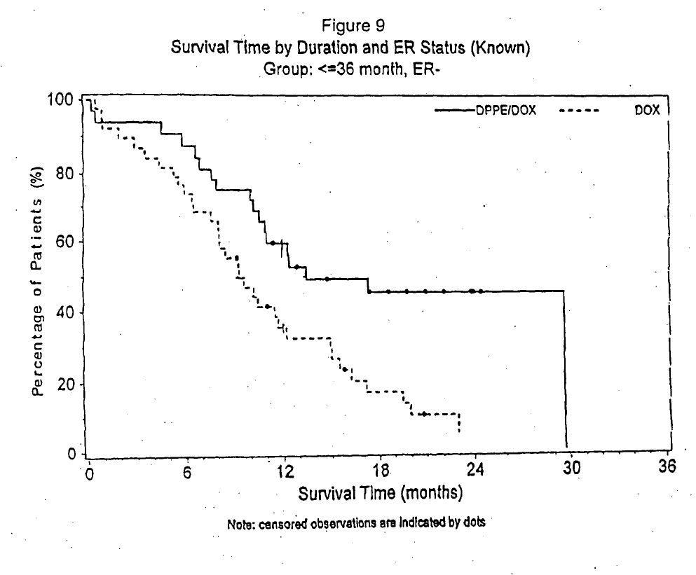

FIG. 10 provides a graphical representation of survival time of ER positive patients having a duration >36 months, receiving DPPE and doxorubicin (DPPE/DOX) or doxorubicin alone (DOX).

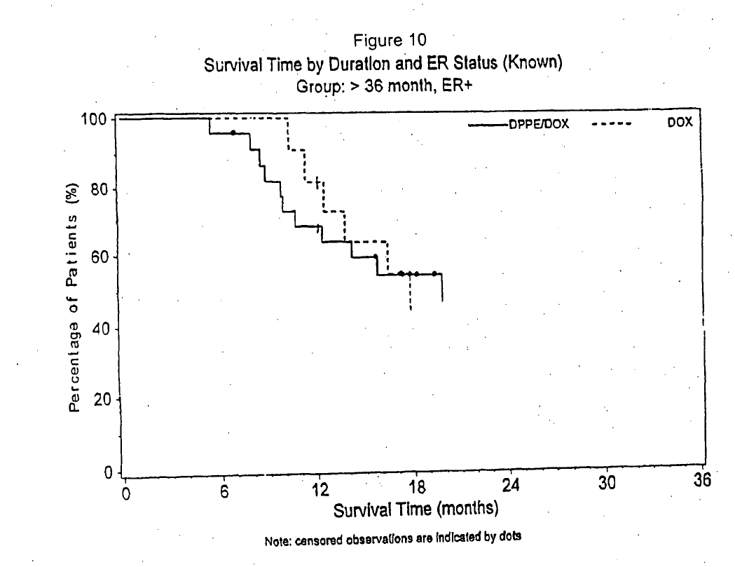

FIG. 11 provides a graphical representation of survival time of ER negative patients having a duration >36 months, receiving DPPE and doxorubicin (DPPE/DOX) or doxorubicin alone (DOX).

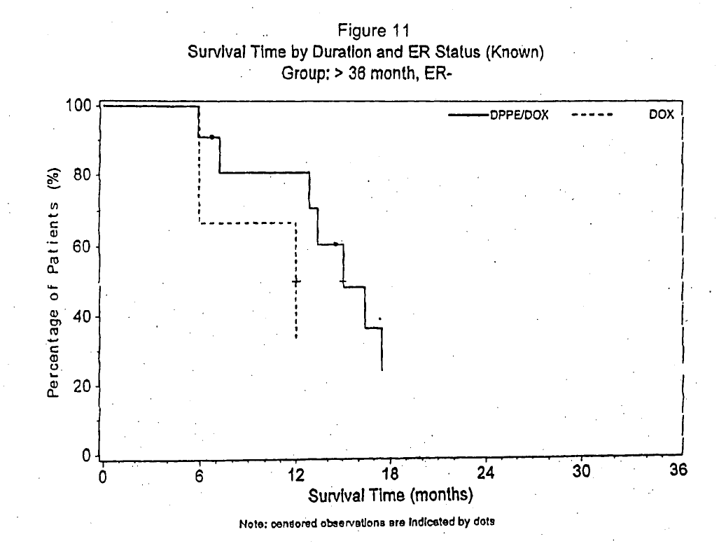

FIG. 12 provides a graphical representation of time to progression for ER negative patients receiving DPPE and doxorubicin (DPPE/DOX) or doxorubicin alone (DOX).

FIG. 13 provides a graphical representation of time to progression for ER positive patients receiving DPPE and doxorubicin (DPPE/DOX) or doxorubicin alone (DOX).

FIG. 14 provides a graphical representation of time to progression for patients having a duration of ≦6 months and receiving DPPE and doxorubicin (DPPE/DOX) or doxorubicin alone (DOX).

FIG. 15 provides a graphical representation of time to progression for patients having a duration of >6 months to ≦36 months and receiving DPPE and doxorubicin (DPPE/DOX) or doxorubicin alone (DOX).

FIG. 16 provides a graphical representation of time to progression for patients having a duration of >36 months and receiving DPPE and doxorubicin (DPPE/DOX) or doxorubicin alone (DOX).

FIG. 17 provides a graphical representation of time to progression for patients receiving DPPE and doxorubicin (DPPE/DOX), with a comparison of patients having a duration of ≦6 months, >6 months to ≦36 months, or >36 months.

FIG. 18 provides a graphical representation of time to progression for patients receiving doxorubicin alone (DOX), with a comparison of patients having a duration of ≦6 months, >6 months to ≦36 months, or >36 months.

FIG. 19 provides a graphical representation of survival lime of patients receiving doxorubicin alone (DOX), with a comparison of patients having a duration of ≦6 months, >6 months to ≦36 months, or >36 months.

FIG. 20 provides a graphical representation of survival time of patients receiving DPPE and doxorubicin (DPPE/DOX), with a comparison of patients having a duration of ≦6 months, >6 months to ≦36 months, or >36 months.

FIG. 21 provides a graphical representation of survival time of patients having a duration of ≦36 months, receiving DPPE and doxorubicin (DPPE/DOX) or doxorubicin alone (DOX). In the DOX arm n=100 and the median is 12.1973. In the DPPE/DOX arm n=91 and the median is 29.6548. p=0.0016.

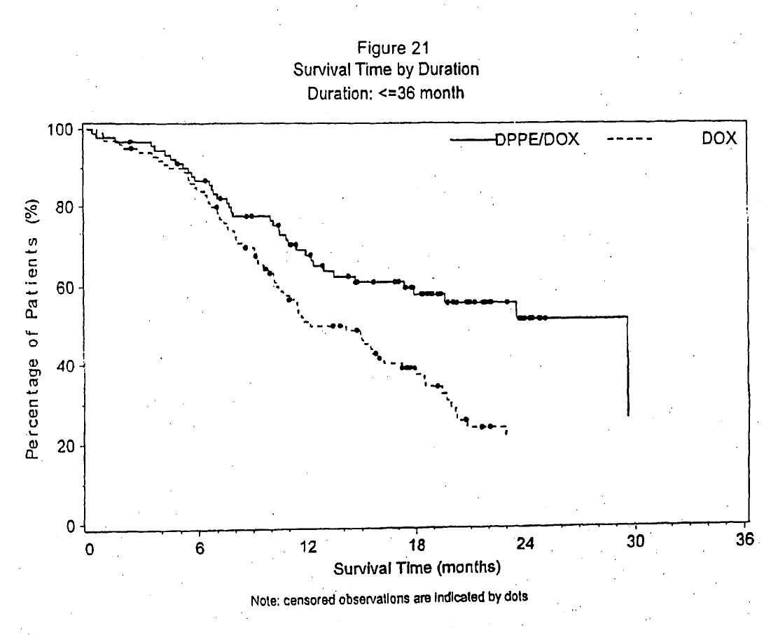

FIG. 22 provides a graphical representation of survival time of patients having a duration >36 months, receiving DPPE and doxorubicin (DPPE/DOX) or doxorubicin alone (DOX). In the DOX arm n=50 and the median is 28.6356. In the DPPE/DOX arm n=62 and the median is 19.8247. p=0.7485.

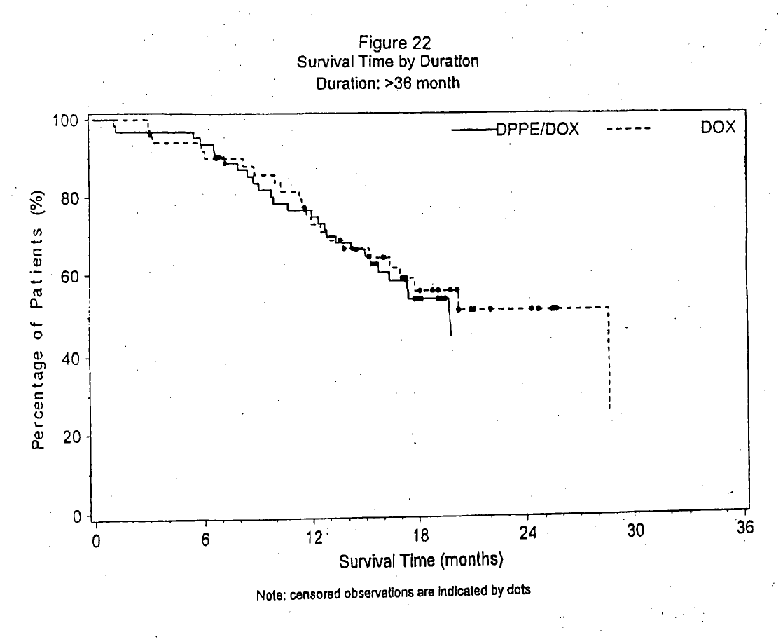

FIG. 23 provides a graphical representation of survival time of patients having a duration of either ≦36 months or >36 months, receiving DPPE and doxorubicin (DPPE/DOX).

FIG. 24 provides a graphical representation of survival time of patients having a duration of either ≦36 months or >36 months, receiving doxorubicin (DOX) alone.

FIG. 25 provides a graphical representation of survival time of patients having a duration of <6 months, receiving DPPE and doxorubicin (DPPE/DOX) or doxorubicin alone (DOX), who had received chemotherapy prior to the trial.

FIG. 26 provides a graphical representation of survival time of patients having a duration of <6 months, receiving DPPE and doxorubicin (DPPE/DOX) or doxorubicin alone (DOX), who had not received chemotherapy prior to the trial.

FIG. 27 provides a graphical representation of survival time of patients having a duration of >6 to ≦36 months, receiving DPPE and doxorubicin (DPPE/DOX) or doxorubicin alone (DOX), who had received chemotherapy prior to the trial.

FIG. 28 provides a graphical representation of survival time of patients having a duration of >6 to ≦36 months, receiving DPPE and doxorubicin (DPPE/DOX) or doxorubicin alone (DOX), who had not received chemotherapy prior to the trial.

FIG. 29 provides a graphical representation of survival time of patients having a duration of >36 months, receiving DPPE and doxorubicin (DPPE/DOX) or doxorubicin alone (DOX), who had received chemotherapy prior to the trial.

FIG. 30 provides a graphical representation of survival time of patients having a duration of >36 months, receiving DPPE and doxorubicin (DPPE/DOX) or doxorubicin alone (DOX), who had not received chemotherapy prior to the trial.

FIG. 31 provides a graphical representation of survival time of patients having a duration of ≦36 months, receiving DPPE and doxorubicin (DPPE/DOX) or doxorubicin alone (DOX), who had received chemotherapy prior to the trial.

FIG. 32 provides a graphical representation of survival time of patients having a duration of ≦36 months, receiving DPPE and doxorubicin (DPPE/DOX) or doxorubicin alone (DOX), who had not received chemotherapy prior to the trial.

### DETAILED DESCRIPTION OF THE INVENTION

The present invention is based on the surprising and unexpected enhanced survival of cancer patients treated with DPPE and a second chemotherapeutic agent in phase III clinical trial studies. This survival advantage was observed in the absence of any preceding statistically significant increase in progression free survival. (PFS) or objective response rate as revealed by interim analysis. Normally, one skilled in the art would expect to see substantial differences in objective response, modest differences in PFS and little, if any, increase in survival, especially in situations in which second or even third line therapy is subsequently available.

In addition, the present invention demonstrates for the first time that specific sub-populations of cancer patients derive a surprising benefit from the survival advantage mediated by DPPE. Notably, in one study breast cancer patients who have a relapse in disease 36 months or less from original breast surgery/diagnosis have been shown to derive the benefit of a DPPE-mediated survival advantage, while patients who originally relapsed after 36 months did not benefit from treatment with DPPE. Other factors, such as estrogen receptor status (i.e. denoting hormone-responsive breast cancers versus hormone-resistant breast cancers) have also been correlated for the first time with the observed DPPE-mediated survival advantage in breast cancer patients.

The identification of such sub-populations allows for the more effective design and delivery of cancer treatments. The present invention provides a method of identifying sub-populations of patients that derive the greatest benefit from DPPE treatment. These sub-populations are identified amongst patients in clinical trials to study the effect of DPPE on a particular cancer. The method involves dividing each arm of the trial (i.e. the DPPE-treated arm and the control arm) into subgroups according to the duration of the cancer, or according to the presence or absence of markers predictive of the aggressivity of the cancer, and analysing the survival time of each sub-group. A statistically significant difference between a subgroup in the DPPE arm compared to the corresponding subgroup in the control arm indicates that a sub-population that derives a benefit from DPPE treatment.

The present invention offers an alternative or supplement to chemotherapy, endocrine therapy and radiation therapy in the treatment of advanced disease in sub-populations of cancer patients thus identified, as well as in the adjuvant setting. Moreover, the present invention offers an alternative or supplement to chemotherapy or tamoxifen therapy for those with estrogen-receptor negative breast cancer.

Definitions

Unless defined otherwise, all technical and scientific terms used herein have the same meaning as commonly understood by one of ordinary skill in the art to which this invention pertains.

“Time-to-progression” or “Progression free survival,” as used herein, refers to the time from the initiation of treatment (or randomisation, as the case may be) to the time of progression, or the time of death for patients who have died in the absence of progression, irrespective of the cause.

“Progression,” as used herein, refers to an increase of at least 25% in the overall sum of measurable lesions as compared to nadir (i.e. best response) and/or the appearance of new lesions.

“Response status,” as used herein, refers to a measurement of the behaviour of a tumour(s) or lesion(s) under chemotherapy, namely any observed growth (progression of disease), stability, or shrinkage (complete or partial response).

“Advanced disease,” as used herein, refers to overt disease in a patient, wherein such overt disease is not amenable to cure by local modalities of treatment, such as surgery or radiotherapy.

“Duration,” as used herein, refers to the time from the initial pathological diagnosis of a primary cancer to the appearance of advanced, metastatic or locally advanced disease which may require institution of chemotherapy (e.g. anthracycline chemotherapy).

“Relapse,” as used herein, refers to the relapse of a patient with advanced disease. “Relapse time,” as used herein, refers to the time from the initial appearance of a primary cancer to the appearance of advanced disease requiring chemotherapy.

“Indolent cancer,” as used herein, refers to a cancer that has relapsed in approximately the latter one third of the spectrum of relapse times for a given cancer. In the case of a breast cancer, “indolent,” as used herein with reference to breast cancer, refers to a cancer that has relapsed after 36 to 40 months following initial diagnosis, wherein the patient has advanced disease and for the first time has become a candidate for chemotherapy (such as anthracycline chemotherapy).

As used herein, the term “aggressive cancer” refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types.

As used herein, the phrase “suspected of having an aggressive cancer,” refers to a situation wherein a patient has had a tumour or lesion, which tumour or lesion had features correlated with the development of advanced disease, for example, markers predictive of aggressive disease. In a specific example, an indication of aggressive breast cancer is a tumour that is estrogen-receptor negative (ER−). Alternatively, the tumour may be ER positive, but the patient may exhibit other markers predictive of aggressive disease, such as node positivity. In these situations adjuvant therapies may be applied.

The term “adjuvant therapy,” as used herein, refers to a treatment that is added to increase the effectiveness of a primary treatment. In cancer, adjuvant therapy usually refers to chemotherapy, hormonal therapy or radiation therapy after surgery (primary therapy) to increase the likelihood of killing all cancer cells.

The term “neoadjuvant therapy,” as used herein, refers to a treatment given before the primary treatment. Examples of neoadjuvant therapy include chemotherapy, radiation therapy, and hormone therapy.

The term “hormone therapy,” as used herein, refers to a treatment in which hormones or anti-hormone drugs are administered to a patient in order to slow or stop the growth of certain cancers (such as prostate and breast cancer) by blocking the body's natural hormones.

The term “hormone-resistant cancer,” as used herein, refers to a cancer that does not respond to hormone therapy, whereas the term “hormone-responsive cancer” refers to a cancer that does respond to hormone therapy.

Therapeutic Use of DPPE

The present invention provides for the use of DPPE in conjunction with one or more other chemotherapeutic agents in the treatment of a patient suffering from cancer in order to enhance survival.

In one embodiment of the present invention, the patient is suffering from a rapidly growing or aggressive cancer. The cancer may be a locally advanced cancer or it may be a metastatic cancer. One skilled in the art will appreciate that when the relapse time is used to define an aggressive cancer, this time will vary depending on the type of cancer and may vary further within sub-populations of patients suffering from the same type of cancer. For example, breast cancer can be considered to be aggressive when the cancer has relapsed within 40 months or less of the initial diagnosis.

In one embodiment of the present invention, DPPE is used to treat an aggressive cancer that has relapsed within a time period of 40 months or less from the time of initial diagnosis. In another embodiment, DPPE is used to treat an aggressive cancer that relapsed within a time period of 38 months or less from the time of initial diagnosis. In another embodiment, DPPE is used to treat an aggressive cancer that relapsed within a time period of 36 months or less. In other embodiments, DPPE is used to treat an aggressive cancer that relapsed within a time period of 34 months or less or 32 months or less the time of initial diagnosis.

Patients who can benefit from DPPE treatment include, but are not limited to, those suffering from leukemias, lymphomas, sarcomas and carcinomas. Specific examples include, but are not limited to, breast cancer, prostate cancer, colorectal cancer, lung cancer, stomach cancer, pancreatic cancer, oesophageal cancer, head and neck cancer, Hodgkin's disease and non-Hodgkin's lymphoma.

The term “leukaemia” refers broadly to progressive, malignant diseases of the blood-forming organs. Leukaemia is typically characterized by a distorted proliferation and development of leukocytes and their precursors in the blood and bone marrow but can also refer to malignant diseases of other blood cells such as erythroleukaemia which affects immature red blood cells. Leukaemia includes, for example, acute nonlymphocytic leukaemia, chronic lymphocytic leukaemia, acute granulocytic leukaemia, chronic granulocytic leukaemia, acute promyelocytic leukaemia, adult T-cell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

The term “sarcoma” generally refers to a tumour which originates in connective tissue, such as muscle, bone, cartilage or fat, and is made up of a substance like embryonic connective tissue and is generally composed of closely packed cells embedded in a fibrillar or homogeneous substance. Sarcomas include bone cancer, soft tissue sarcomas, chondrosarcoma, fibrosarcoma, lymphosarcoma, melanosarcoma, myxosarcoma, osteosarcoma, osteogenic sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumour sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented haemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, spindle cell sarcoma and telangiectaltic sarcoma.

The term “carcinoma” refers to a malignant new growth made up of epithelial cells tending to infiltrate the surrounding tissues and give rise to metastases. Exemplary carcinomas include, for example, bladder cancer, breast cancer, cervical cancer, colon cancer, kidney cancer, liver cancer, lung cancer, ovarian cancer, pancreatic cancer, prostate cancer, rectal cancer, skin cancer, stomach cancer, thyroid cancer, uterine cancer, cancer of the vulva, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colorectal carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, haematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, non-small cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telanglectaticum, carcinoma telangiectodes, transitional cell carcinoma, carcinoma tuberosum, tuberous carcinoma, verrucous carcinoma, and carcinoma villosum.

Additional cancers encompassed by the present invention include, for example, multiple myeloma, neuroblastoma, rhabdomyosarcoma, primary thrombocytosis, primary macroglobulinemia, small-cell lung tumours, primary brain tumours, malignant pancreatic insulanoma, malignant carcinoid, gliomas, testicular cancer, genitourinary tract cancer, malignant hypercalcemia, endometrial cancer and adrenal cortical cancer.

Adenocarcinomas are carcinomas that originate in cells that make organs which have glandular (secretory) properties or that originate in cells that line hollow viscera, such as the gastrointestinal tract or bronchial epithelia. For example, breast cancer, prostate cancer, and the like are adenocarcinomas. In one embodiment of the present invention, DPPE in conjunction with one or more other chemotherapeutic agents is used to treat patients with adenocarcinomas. In another embodiment, the adenocarcinoma is breast cancer. In another embodiment, the breast cancer is an aggressive cancer. In another embodiment, the breast cancer is a locally advanced cancer. In still another embodiment, the breast cancer is metastatic breast cancer.

In another embodiment of the present invention, DPPE either alone, or in combination with one or more other chemotherapeutic agents, is used to treat a breast cancer patient with advanced disease within 40 months or less from the time of diagnosis. In another embodiment, DPPE is used to treat the breast cancer patient within 36 months or less from the time of diagnosis.

As is known in the art, cancers that originate in endocrine glands, such as breast and prostate cancer, can be resistant or responsive to hormone therapy. Hormone-resistant cancers are typically more aggressive than their hormone-responsive counterparts. The present invention contemplates the use of DPPE in conjunction with one or more other chemotherapeutic agents, to treat both hormone-resistant and hormone-responsive cancers.

Hormone-resistant breast cancers are known to lack a functional estrogen receptor (ER). Thus, in one embodiment of the invention, DPPE is used to treat ER-negative (ER−) breast cancers. In another embodiment, DPPE is used to treat ER-positive (ER+) breast cancers. The term “estrogen-receptor negative (ER−) breast cancer” is used herein to denote the disorder of those patients who have ER− breast cancer tumours prior to primary treatment and the term “estrogen-receptor positive (ER+) breast cancer” is used herein to denote the disorder of those patients who have ER+ breast cancer tumours prior to primary treatment. Methods of classifying tumours as ER+ or ER− are well-known to those skilled in the art and include, but are not limited to, measurement of intracellular receptor protein by either a steroid-binding assay or by immunochemical assay or by measuring mRNA corresponding to the receptor protein using Northern blot analysis.

The present invention contemplates that DPPE in conjunction with one or more chemotherapeutic agents, may be used as part of a neoadjuvant therapy or as part of an adjuvant therapy to treat a patient suspected of having an aggressive cancer. Alternatively, DPPE may be used in conjunction with one or more chemotherapeutic agents to treat a recurring and/or aggressive cancer, metastatic or advanced disease. DPPE can be used to treat patients who have undergone prior chemotherapy or it may be used to treat chemotherapy naïve patients. Thus, in one embodiment of the invention, DPPE is used as part of an adjuvant therapy. In another embodiment, DPPE is used as a second line of therapy. In another embodiment, DPPE is used to treat patients who have already undergone one or more courses of prior chemotherapy.

In an adjuvant or neoadjuvant setting, it will not be readily apparent whether or not a patient has an aggressive cancer or advanced disease. A variety of markers are known in the art, the presence of which in relation to a tumour is predictive of aggressivity or advanced disease. One or more of these markers are suitable for use in the evaluation of patients suspected of having an aggressive cancer in order to determine whether the cancer is aggressive and thus whether the patient would benefit from the use of DPPE as part of a neoadjuvant or adjuvant therapy. For example, breast cancers that are estrogen-receptor negative (ER−) are highly likely to be aggressive breast cancers. It is also known, however, that ER+ breast cancers can be aggressive. A patient with an ER+ cancer, therefore, can be further evaluated by determination of the presence or absence of other markers, such as node positivity, the presence of which is widely accepted to be an indicator of aggressive disease.

In one embodiment of the present invention. DPPE is used as part of a neoadjuvant or adjuvant therapy in the treatment of a patient with a breast cancer that is ER−. In another embodiment. DPPE is used as part of a neoadjuvant or adjuvant therapy in the treatment of a patient with a breast cancer that is ER+ and who exhibits node positivity.

As indicated above. DPPE is used in conjunction with one or more chemotherapeutic agents. A wide range of cancer chemotherapeutic agents is known in the art and includes those chemotherapeutic agents which are specific for the treatment of a particular type of cancer as well as those which may be applicable to a range of cancers, such as doxorubicin, mitoxantrone, irinotecan (CPT-11). The present invention contemplates the use of both types of chemotherapeutic agent in conjunction with DPPE. Combination therapies using standard cancer chemotherapeutics are also well known in the art and may be used in conjunction with DPPE. Examples of chemotherapeutic agents suitable for the treatment of breast cancer include, but are not limited to, cyclophosphamide, ifosfamide, cisplatin, carboplatin, 5-fluorouracil (5-FU), taxanes such as paclitaxel and docetaxel and various anthracyclines, such as doxorubicin and epi-doxorubicin (also known as epirubicin). Combination therapies using standard cancer chemotherapeutics may also be used in conjunction with DPPE and are also well known in the art, for example, the combination of epirubicin with paclitaxel or docetaxel, or the combination of doxorubicin or epirubicin with cyclophosphamide, which are used for breast cancer treatments. Polychemotherapeutic regimens are also useful and may consist, for example, of doxorubicin/cyclophosphamide/5-fluorouracil or cyclophosphamide/epirubicin/5-fluorouracil.

Cyclophosphamide, mitoxantrone and estramustine are known to be suitable for the treatment of prostate cancer. Cyclophosphamide, vincristine, doxorubicin and etoposide are used in the treatment of small cell lung cancer, as are combinations of etoposide with either cisplatin or carboplatin. In the treatment of stomach or oesophageal cancer, combinations of doxorubicin or epirubicin with cisplatin and 5-fluorouracil are useful. For colorectal cancer, CPT-11 alone or in combination with 5-fluorouracil-based drugs, or oxaliplatin in combination with 5-fluorouracil-based drugs can be used. Other examples include the combination of cyclophosphamide, doxorubicin, vincristine and prednisone in the treatment of non-Hodgkin's lymphoma; the combination of doxorubicin, bleomycin, vinblastine and DTIC in the treatment of Hodgkin's disease and the combination of cisplatin or carboplatin with any one or a combination of gemcitabine, paclitaxel, docetaxel, vinorelbine or etoposide in the treatment of non-small cell lung cancer.

In one embodiment of the present invention, DPPE is used in combination with an anthracycline, such as doxorubicin or epirubicin, either with or without other chemotherapeutics. In another embodiment, DPPE is used in combination with a taxane, either with or without other chemotherapeutics.

Pharmaceutical Compositions

The synthesis of DPPE and its salts has been described in the art, for example, see U.S. Pat. No. 4,803,227. The pharmaceutically active compound or salts thereof may be administered as pharmaceutical compositions with an appropriate pharmaceutically physiologically acceptable carrier, diluent, excipient or vehicle. The pharmaceutical compositions may also be formulated to contain DPPE and one or more other chemotherapeutic agents for concurrent administration to a patient.

The pharmaceutical compositions of the present invention may be administered orally, topically, parenterally, by inhalation or spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

The pharmaceutical compositions may be in a form suitable for oral use, for example, as tablets, troches, lozenges, aqueous or oily suspensions, dispersible powders or granules, emulsion hard or soft capsules, or syrups or elixirs. Compositions intended for oral use may be prepared according to methods known to the art for the manufacture of pharmaceutical compositions and may contain one or more agents selected from the group of sweetening agents, flavouring agents, colouring agents and preserving agents in order to provide pharmaceutically elegant and palatable preparations. Tablets contain the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed.

Pharmaceutical compositions for oral use may also be presented as hard gelatine capsules wherein the active ingredient is mixed with an inert solid diluent, for example, calcium carbonate, calcium phosphate or kaolin, or as soft gelatine capsules wherein the active ingredient is mixed with water or an oil medium such as peanut oil, liquid paraffin or olive oil.

Aqueous suspensions contain the active compound in admixture with suitable excipients including, for example, suspending agents, such as sodium carboxymethylcellulose, methyl cellulose hydropropylmethylcellulose, sodium alginate, polyvinylpyrrolidone, gum tragacanth and gum acacia; dispersing or wetting agents such as a naturally-occurring phosphatide, for example, lecithin, or condensation products of an alkylene oxide with fatty acids, for example, polyoxyethyene stearate, or condensation products of ethylene oxide with long chain aliphatic alcohols, for example, hepta-decaethyleneoxycetanol, or condensation products of ethylene oxide with partial esters derived from fatty acids and a hexitol for example, polyoxyethylene sorbitol monooleate, or condensation products of ethylene oxide with partial esters derived from fatty acids and hexitol anhydrides, for example, polyethylene sorbitan monooleate. The aqueous suspensions may also contain one or more preservatives, for example ethyl, or n-propyl p-hydroxy-benzoate, one or more colouring agents, one or more flavouring agents or one or more sweetening agents, such as sucrose or saccharin.

Oily suspensions may be formulated by suspending the active ingredients in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid.

Dispersible powders and granules suitable for preparation of an aqueous suspension by the addition of water provide the active compound in admixture with a dispersing or wetting agent, suspending agent and one or more preservatives. Suitable dispersing or wetting agents and suspending agents are exemplified by those already mentioned above. Additional excipients, for example sweetening, flavouring and colouring agents, may also be present.

Pharmaceutical compositions of the invention may also be in the form of oil-in-water emulsions. The oil phase may be a vegetable oil, for example, olive oil or arachis oil, or a mineral oil, for example, liquid paraffin, or it may be a mixtures of these oils. Suitable emulsifying agents may be naturally-occurring gums, for example, gum acacia or gum tragacanth; naturally-occurring phosphatides, for example, soy bean, lecithin; or esters or partial esters derived from fatty acids and hexitol, anhydrides, for example, sorbitan monoleate, and condensation products of the said partial esters with ethylene oxide, for example, polyoxyethylene sorbitan monoleate. The emulsions may also contain sweetening and flavouring agents.

Syrups and elixirs may be formulated with sweetening agents, for example, glycerol, propylene glycol, sorbitol or sucrose. Such formulations may also contain a demulcent, a preservative, and/or flavouring and colouring agents.

The pharmaceutical compositions may be in the form of a sterile injectable aqueous or oleaginous suspension. This suspension may be formulated according to known art using suitable dispersing or wetting agents and suspending agents such as those mentioned above. The sterile injectable preparation may also be sterile injectable solution or suspension in a non-toxic parentally acceptable diluent or solvent, for example, as a solution in 13-butanediol. Acceptable vehicles and solvents that may be employed include, but are not limited to, water, Ringer's solution, lactated Ringer's solution and isotonic sodium chloride solution. Other examples are, sterile, fixed oils which are conventionally employed as a solvent or suspending medium, and a variety of bland fixed oils including, for example, synthetic mono- or diglycerides. In addition, fatty acids such as oleic acid find use in the preparation of injectables.

Other pharmaceutical compositions and methods of preparing pharmaceutical compositions are known in the art and are described, for example, in “Remington: The Science and Practice of Pharmacy,” Gennaro, A., Lippincott, Williams & Wilkins, Philidelphia, Pa. (2000) (formerly “Remingtons Pharmaceutical Sciences”).

Administration and Dosage Protocols

In accordance with the present invention, DPPE or a pharmaceutical composition comprising DPPE is administered to a patient in order to treat an aggressive cancer. DPPE or a pharmaceutical composition comprising DPPE may be administered in a manner consistent with its normal manner of administration following conventional chemotherapeutic practice. Typically DPPE is administered as a solution by intravenous infusion.

In one embodiment of the present invention, DPPE is administered to the patient in conjunction with one or more chemotherapeutic agents. DPPE can be administered prior to, or after, administration of the one or more other chemotherapeutic agents, or it can be administered concomitantly.

When DPPE is administered prior to the one or more other chemotherapeutic agents, the length of time between administration of the DPPE and the other compound(s) will depend on the mode of administration and the size of the patient. Generally, DPPE is administered to the patient for between about 30 minutes and about 90 minutes prior to administration of the other chemotherapeutic agent(s). In one embodiment, DPPE is administered to the patient for about 60 minutes prior to administration of the other chemotherapeutic agent(s).

When DPPE and the one or more other chemotherapeutic agents are administered concurrently, administration of the compounds may be initiated at the same time, or administration of the other chemotherapeutic(s) may be initiated at a suitable time after administration of DPPE was initiated. Generally, administration of the other chemotherapeutic(s) is initiated about 30 minutes to about 90 minutes after administration of DPPE was initiated. In one embodiment of the present invention, administration of the other chemotherapeutic(s) is initiated about 60 minutes after administration of DPPE was initiated.

The dosage of DPPE to be administered will be dependent upon the type of cancer to be treated and the size of the patient and can be readily determined by a skilled practitioner. DPPE dosages of 4 mg/kg (160 mg/M²) administered over 1 hour (intravenously) have been shown to be non-toxic and dosages of only 8 mg/M² over 24 to 72 hours do not result in clinical side effects, while dosages of 240 mg/M² administered over 1 hour may result in CNS toxicity (see, for example, U.S. Pat. No. 5,859,065). Typically, the dosage range of DPPE is between about 8 mg/M² and about 320 mg/M². In some instances, however, dosages up to 1200 mg/M² per day may be appropriate.

In one embodiment of the present invention, a DPPE dosage of between 8 mg/M² and 240 mg/M² is administered to a patient over a time period of 30 minutes to 90 minutes. In another embodiment, a DPPE dosage of between 4 mg/kg and 8 mg/kg is administered to a patient over a time period of 80 minutes. In other embodiments, a DPPE dosage of about 6 mg/kg (or 240 mg/M²) or of about 5.3 mg/kg is administered to a patient over a time period of 80 minutes. In another embodiment, the DPPE is administered with concurrent administration of one or more other chemotherapeutic agents over the last 20 minutes. In still another embodiment, the other chemotherapeutic is doxorubicin. In a related embodiment, the doxorubicin is administered at a dose of 60-90 mg/M².

Treatment regimens are typically designed such that the DPPE is administered to the patient in cycles. Treatment with DPPE in accordance with the present invention may be part of a treatment regimen that involves one cycle of administration or the regimen may involve more than one cycle. Generally, the treatment regimen involves between about 2 and about 10 cycles. In one embodiment of the present invention, the treatment regimen involves between about 2 and about 8 cycles. In another embodiment, the treatment regimen involves about 4 cycles. Typically, a cycle is between about 1 and about 4 weeks. In one embodiment, the cycle is about 3 weeks.

If cyclophosmamide were to be included as a chemotherapeutic with DPPE in such a treatment regimen, it could be administered at a dose of approximately 600 mg/M². It is to be understood, however, that the dosage and frequency of administration may be adapted to the circumstances in accordance with known practices in the art, for the treatment of different cancers.

Pharmaceutical Kits

The present invention additionally provides for therapeutic kits containing DPPE in pharmaceutical compositions for use in the treatment of cancer. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

When the components of the kit are provided in one or more liquid solutions, the liquid solution can be an aqueous solution, for example a sterile aqueous solution. In this case the container means may itself be an inhalant, syringe, pipette, eye dropper, or other such like apparatus, from which the composition may be administered to a patient.

The components of the kit may also be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or any such medically approved delivery vehicle.

The kit may further comprise one or more other chemotherapeutic agents for administration to a patient in conjunction with DPPE.

To gain a better understanding of the invention described herein, the following examples are set forth. It should be understood that these examples are for illustrative purposes only. Therefore, they should not limit the scope of this invention in any way.

### EXAMPLES

A phase III clinical trial study (the results of which are analyzed in the examples which follow) was conducted which involved breast cancer patients who relapsed following surgery, requiring anthracycline chemotherapy. Following surgery and prior to inclusion in the trial, patients may or may not have received treatments, such as radiotherapy, hormone treatments or other chemotherapies, but not treatment with anthracyclines.

Two arms of treatment were involved in this trial. Patients were randomly selected to participate in either arm. In one arm, patients were treated with DPPE and doxorubicin, in the other arm patients were treated with doxorubicin alone.

In the trial, patients were permitted to stay on doxorubicin for a maximum of about 7 cycles (5 months). Many of the patients stopped DPPE before stopping doxorubicin, thus the median number of DPPE cycles was 4 (i.e. 3 months, range 1 to 8 cycles), whereas the median number of doxorubicin cycles on the DPPE arm was 6 (i.e. 4½ months, range 1 to at least 8 cycles). DPPE was administered to patients in the amount of 6 mg/kg (or 240 mg/M²) over 80 minutes, with the concurrent administration of doxorubicin over the last 20 minutes at a dose of 60-90 mg/M².

### Example I

### Demonstration of DPPE-Mediated Survival Advantage

Between 3 and 8 months following the start of treatment of patients, only a small and non-widening difference was observed between the DPPE survival and control curves. At about 8 months, however, the DPPE curve trajectory ceases to be parallel with the control curve and quite clearly starts to flatten its trajectory. This abrupt inflexion in the curve at 8 months indicates that there is a reduction in the rate of death due to breast cancer, which is suggestive of a DPPE-related survival advantage. In short, breast cancer patients on standard chemotherapy lived on average fifteen months and those who had taken DPPE together with standard chemotherapy lived on average just over 23 months. Furthermore, many of the patients in the DPPE arm are still alive, and fewer of the control group are still alive. Therefore, the true extent of the survival difference would be expected to increase.

### Example II

### Effect of DPPE on Patients with Estrogen-Receptor Negative Cancers

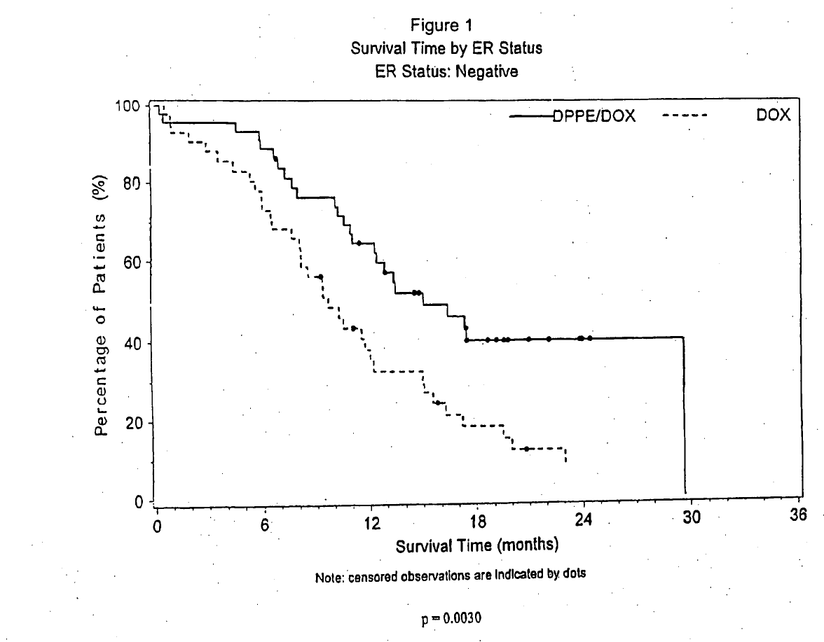

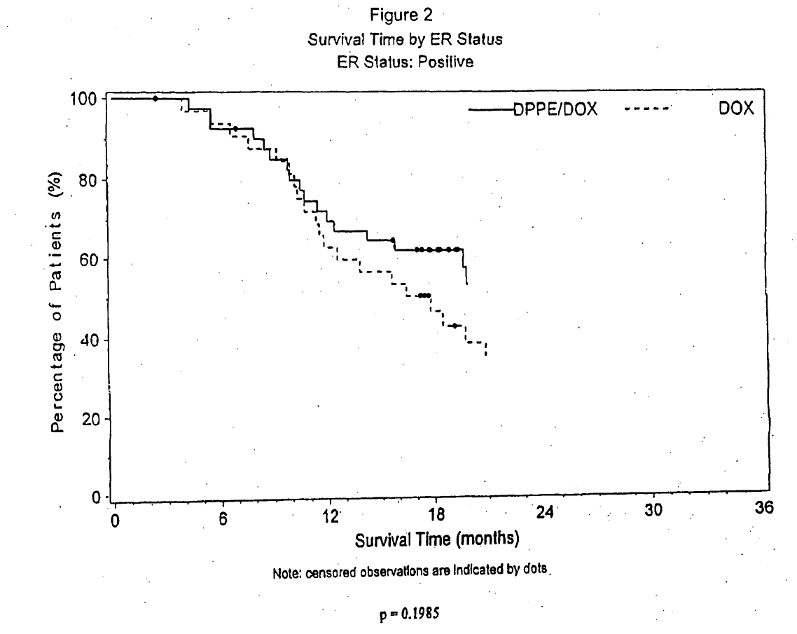

The survival analysis by subgroup (see FIGS. 1, 2, 3 and 4) indicates a benefit in the known ER− patients due to DPPE. A benefit is also observed in ER+ patients, which is less and appears later (at about 11 months). ER analysis shows the control ER+/− difference being obscured by DPPE thus increasing the ER− significantly, and the ER+ only a little. Overall, many of the patients with indolent cancers (>36 m duration) are in fact ER+, and DPPE seems mainly to help the patients with ER− breast cancer, a more rapidly progressive disease.

ER status was only ascertained in about half the sample (see Table 2). By analysing whether endocrine therapy was received prior to trial, one can impute an ER status to the unknowns, such that those receiving hormone treatment are regarded as ER+ and those not receiving it are regarded as ER−.

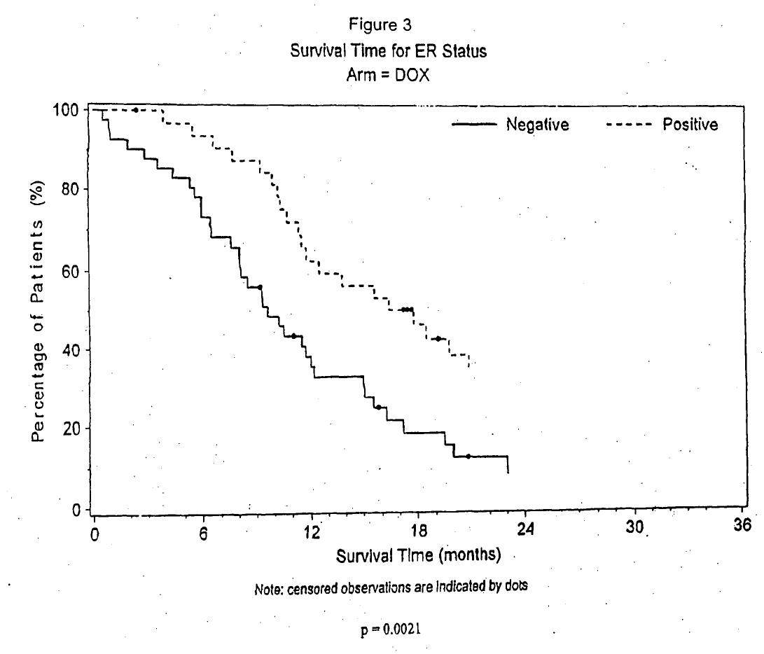

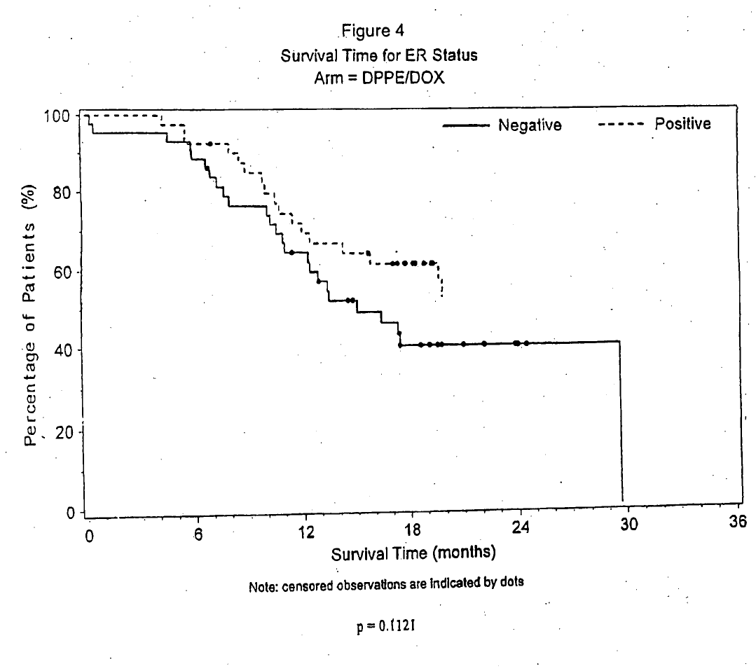

This ER− difference is very large (p=0.003) and the p value is the most impressive in any subgroup, all the more so for being achieved with only half the sample. FIGS. 3 and 4 show a big difference between ER+ (longer) and ER− (shorter) survival for the DOX arm (p=0.0021), which shrinks dramatically under the influence of DPPE (p=0.1121) due to the relative improvement in ER− patients.

Table 1 shows that in each group the ER+ patients did better than the ER− patients, but this difference is large for the control group and marginal for the DPPE group. Furthermore, the reason for the obscuration of the ER+/− survival difference relates to the huge improvement in ER− survivals in the DPPE arm. The very asymmetric effect by ER status points to an underlying biological reality, which in turn is further evidence of the survival advantage mediated by DPPE in a distinct sub-population of breast cancers.

Including both ER known and imputed (i.e. the whole sample) does not change the results substantially: the p value for ER− survival is 0.0054 (with 81 ER− patients in the DOX arm and 85 in the DPPE/DOX arm); the p value for ER+ survival is 0.3934 (with 69 in DOX and 68 in DOX/DPPE). Interestingly, for time-to-progression (TTP) the ER+ graph achieves significance (p=0.0445). These data further demonstrate that ER+ tumours may be helped by DPPE.

### Example III

### Effect of DPPE on Patients Diagnosed with Cancer in 3 Years or Less Post Surgery

The patients in the trial were assigned to subgroups based on duration, i.e. the length of time between the initial diagnosis of breast cancer and the appearance of advanced disease. The subgroups were: (i) less than or equal to 6 months, (ii) between 6 months and 36 months and (iii) more than 36 months.

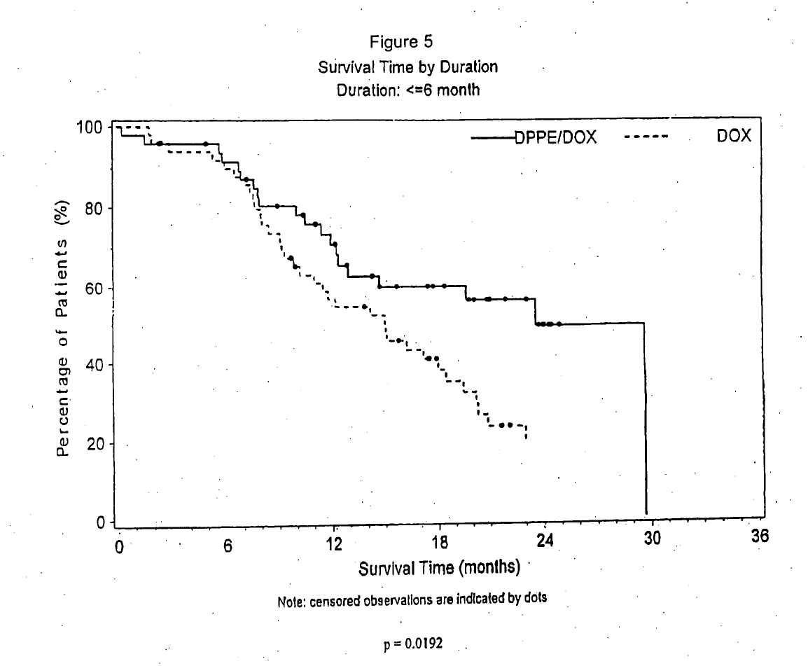

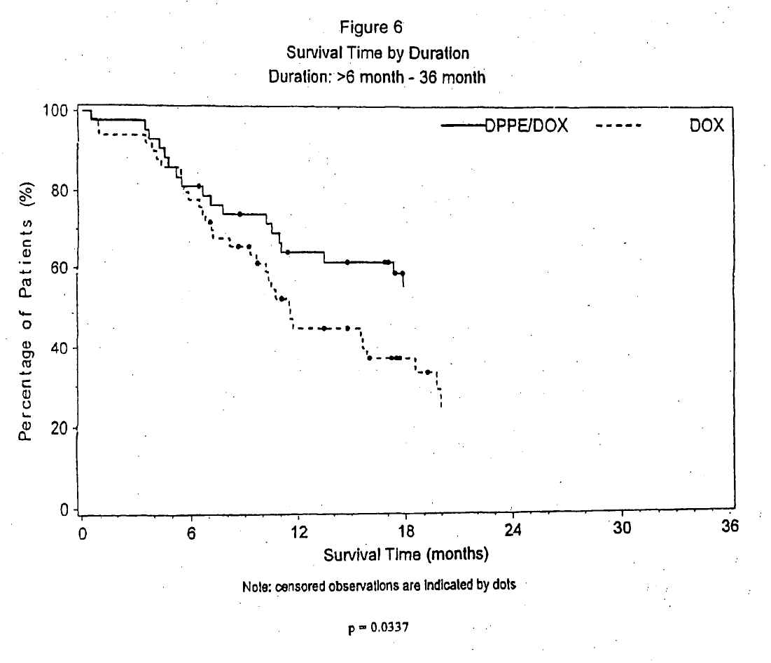

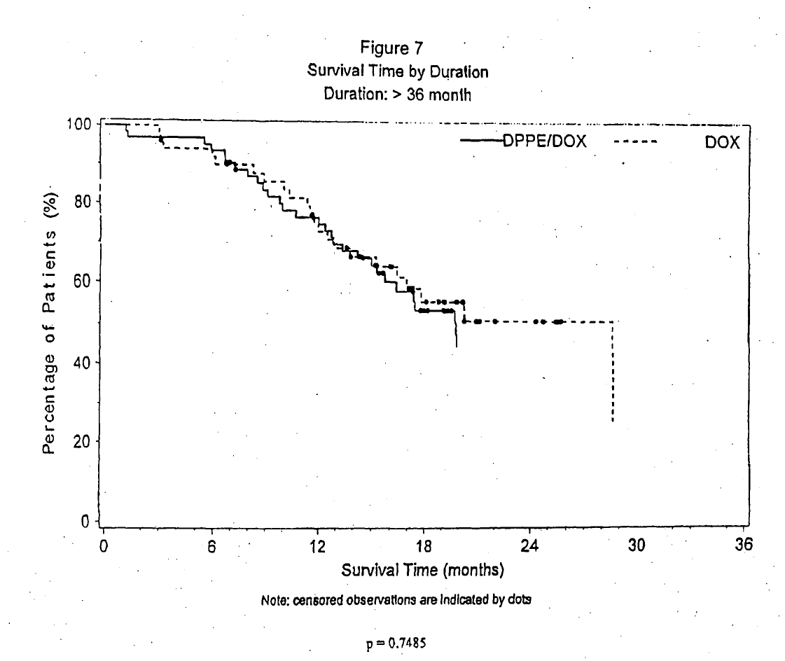

Analysis of survival by duration subgroup revealed large differences for the < or = to 6 months, and 6 to 36 month subgroup in favour of DPPE, but there appears to be no difference in those patients whose duration was >36 months (FIGS. 5, 6 and 7, respectively). These results ate shown in Table 3.

These data show statistically that patients with more rapidly relapsing disease had substantial benefit whereas those with more indolent disease had no benefit. Note that 191/303patients (63%) relapsed in <36 months, so that the sub-population which benefited from the DPPE mediated survival advantage accounted for about two thirds of the patients in the trial and by analogy about two-thirds of possible breast cancer patients with advanced disease.

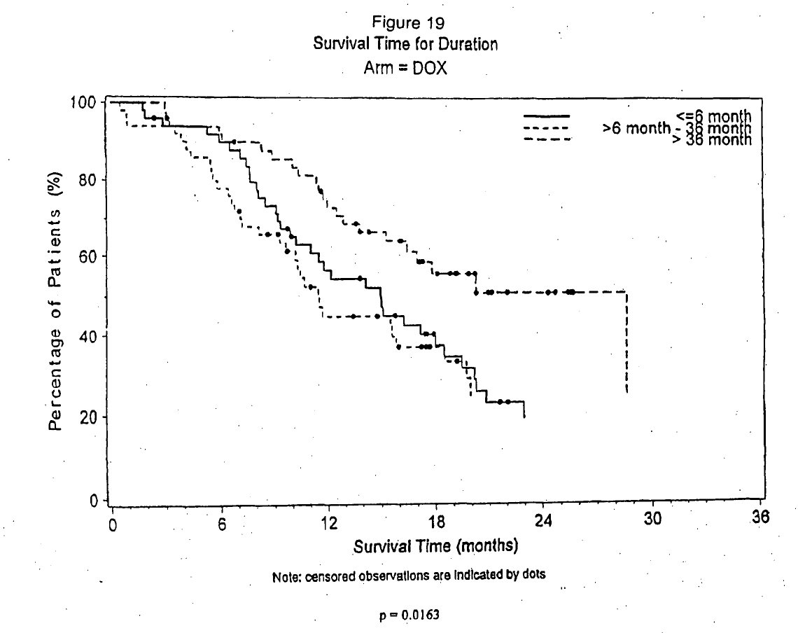

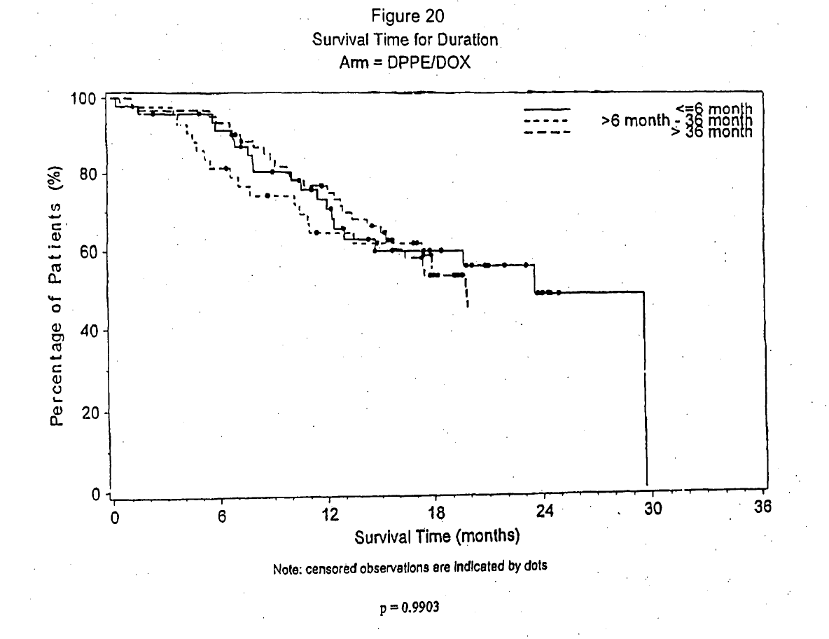

In the doxorubicin alone arm (DOX), there is a spread amongst the survival times for the 3 duration subgroups, which is statistically significant (p=0.0163; see FIG. 19) with the more benefit being derived by the patients with indolent cancers (36 months) than by either of the two shorter duration groups (which are similar). However, in the DPPE/DOX group, the 3 subgroups by duration show similar survival times and the p value is lost (0.9903; see FIG. 20) because the two shorter duration groups now behave very much like the longer duration (indolent) group. In this regard, indolent patients are different from those that have short duration times and do not appear to benefit from treatment with DPPE.

There is no significant difference in the effect of DPPE observed in patients who had a relapse in disease <6 m and 6-36 m post diagnosis (see FIG. 20). These two sub-populations may accordingly be regarded as one group.

It is important to note that the optimal split in duration between aggressive and indolent cancer may be different for different cancers, or within sub-populations of a particular group of cancer patients. For example, it may be that the optimal cut-off point for DPPE efficacy in breast cancer patients is 32 months, 40 months, some other month in-between, or within the range between 32 and 40 months. Furthermore, the optimal cut-off point or range may vary for ER− and ER+ patients. The optimal split in a given instance may be found by dichotomising patients in clinical trials along various time points e.g. <32 m vs. ≧32 m; <33 m vs. ≧33 m; etc.

Time-to-Progression (TTP) by Duration

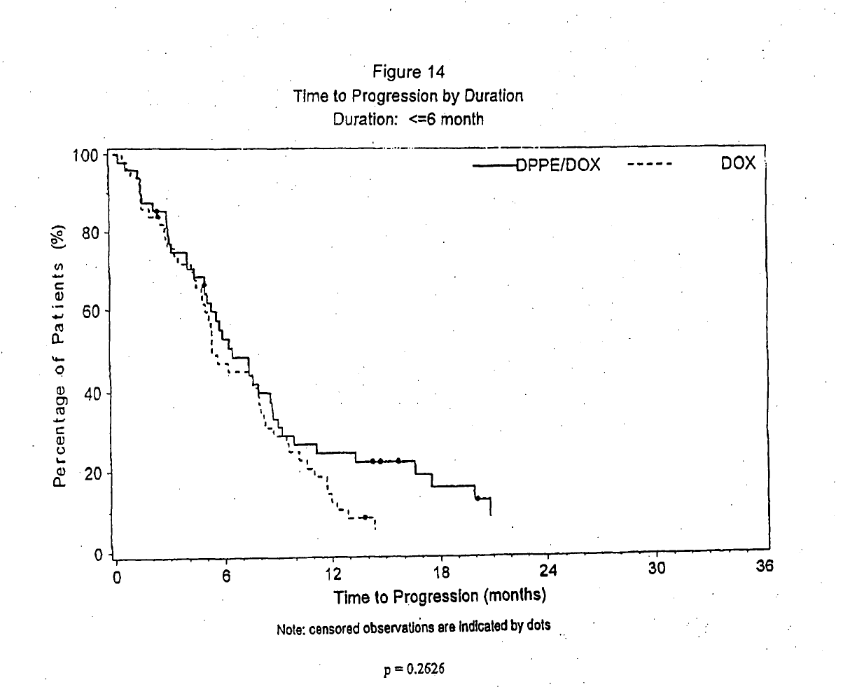

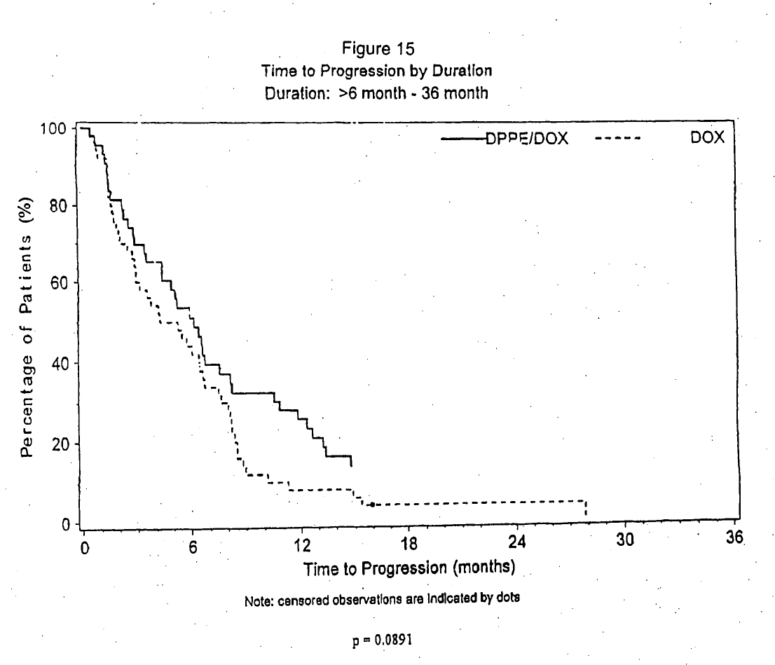

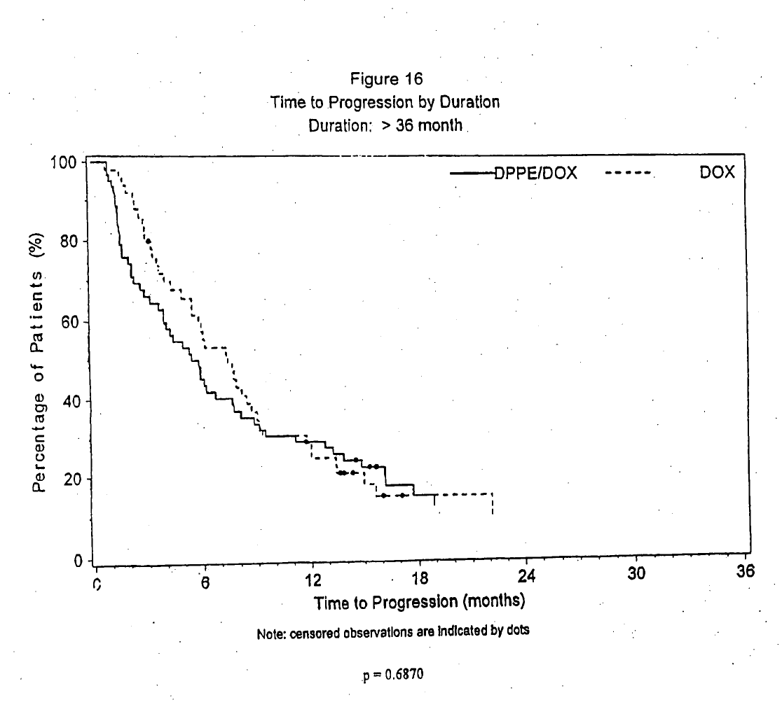

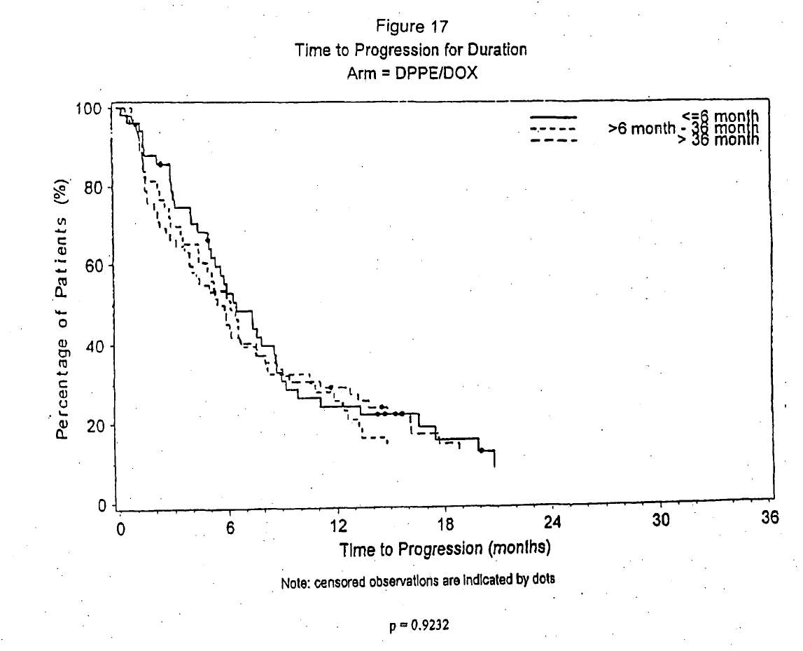

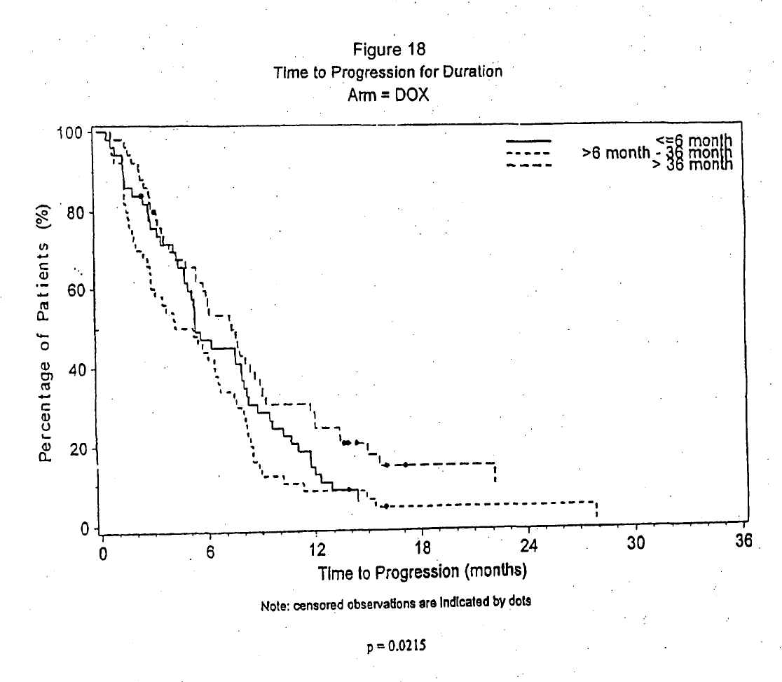

The p values for TTP by duration indicate a correlation with the survival by duration outcomes. By inspection, the curves for the <6 m and 6-36 m subgroups do exhibit a break-apart at about 8 to 9 m (see FIGS. 14, 15 and 16). For the indolent >36 m subgroup, there is no break-apart. This pattern of correlation with survival by duration, and the p values, suggest that whatever was responsible for the survival prolongation was operating during the co-administration of DOX and DPPE. Further corroboration for this conclusion comes from the graphs provided in FIGS. 17 and 18 which show that the statistically significant spread of TTP by the 3 subgroups (duration) in the DOX arm (p=0.0215; FIG. 17) is lost in the DOX/DPPE arm (p=0.9232; FIG. 18). Again, this is due to the improvement in the two shorter relapse subgroups, such that under the influence of DPPE the TTP course for these two groups very closely resembles that of the indolent group.

It is to be understood that the 36 m time point used for the purposes of the illustrated embodiment of the invention is only one specific duration marker for the efficacious administration of DPPE to cancer patients. As indicated above, the cut-off for determining which cancers are aggressive and which are indolent may be earlier or later than the value selected for this trial.

### Example IV

### Survival and Time-to-Progression by Response Status

In the present study, survival and TTP data is presented for the first time as a function of response to chemotherapy. The data presented in Table 4 indicate that patients who respond to doxorubicin probably benefit from the addition of DPPE (p=0.1201) and those whose disease stabilises show definite benefits (p=0.0075) (see also FIGS. 5, 6 and 7). By contrast, those who experience immediate disease progression experience no benefit (p=0.9232).

Accordingly, overall, it seems that benefit from DPPE is concentrated in patients whose disease responds or stabilises on doxorubicin, who are ER− and who relapse within 36 m. However, benefit in ER+ patients is also seen.

### Example V

### Survival Time by Duration and Estrogen-Receptor Status

Tables 5 and 6 demonstrate that the proportion of ER− patients is relatively constant over the two shorter relapsing subgroups (≦6 m and 6-36 m patients), at 67.9% and 60.7%, but that the proportion of ER− patients in the ≧36 m indolent subgroups to 29.2%. This pattern is consistent with ER status being a significant and useful marker of the DPPE-mediated survival advantage.

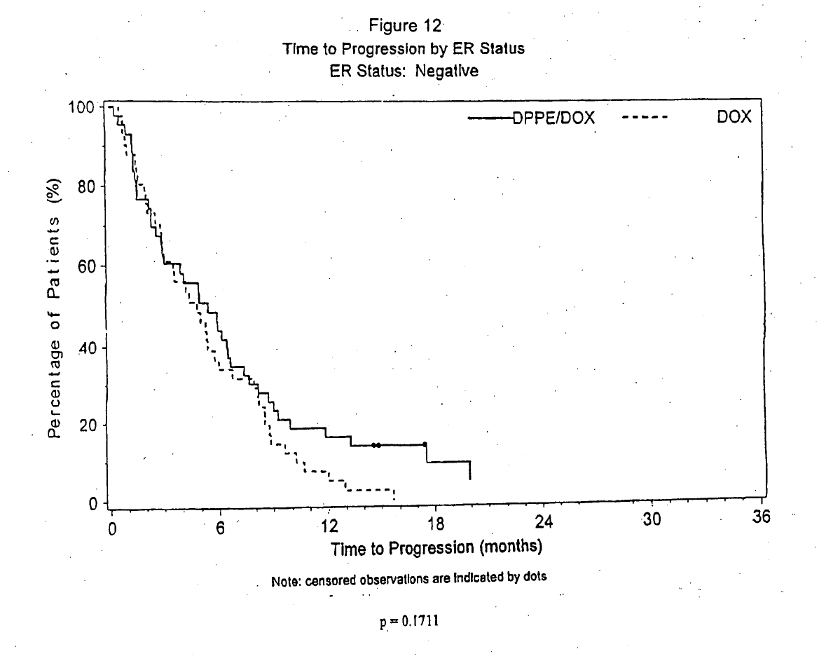

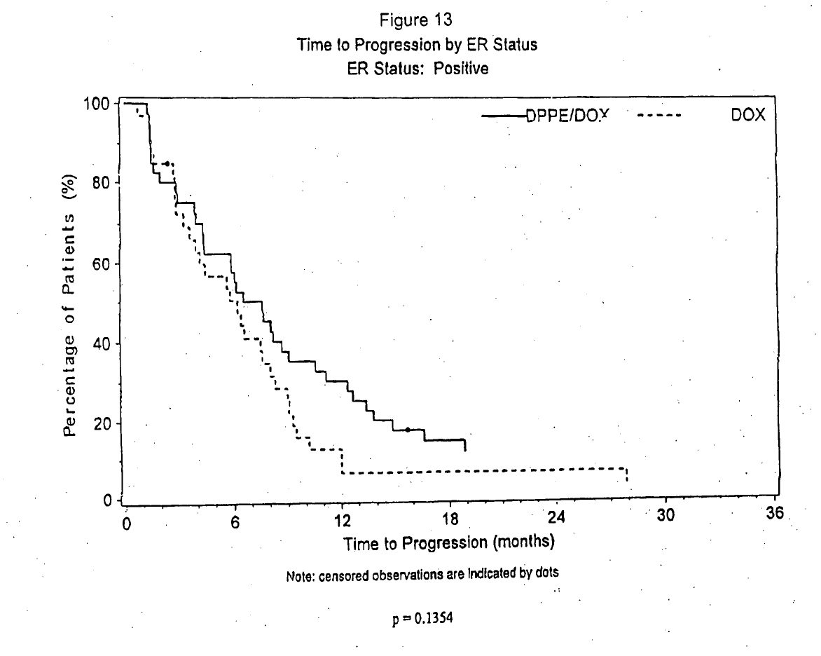

Closer examination of the short duration (i.e. <36 m) ER+ patients and the long duration (i.e. ≧36 m) ER− patients (Table 7, note that the two shorter relapsing groups (i.e. ≦6 m and 6-36 m) have been fused into one <36 m group), suggests that it is not ER which is the key driver of the DPPE-mediated effects, but duration. The <36 m, ER− subgroup shows a large difference, as expected. The >36 m ER+ subgroup shows no difference, as expected. The >36 m, ER− subgroup has too few number to be meaningful. When one looks at this subgroup with imputed values added in, the p value is 0.9008 indicating that there is little difference. This analysis suggests that duration is probably a bigger factor than ER status and that ER+ patients with duration <36 m also appear to benefit from DPPE treatment. TTP data is also consistent with this conclusion indicating some benefit for ER+ patients (see FIGS. 12 and 13).

### Example VI

### Survival Time by Duration and Treatment Type

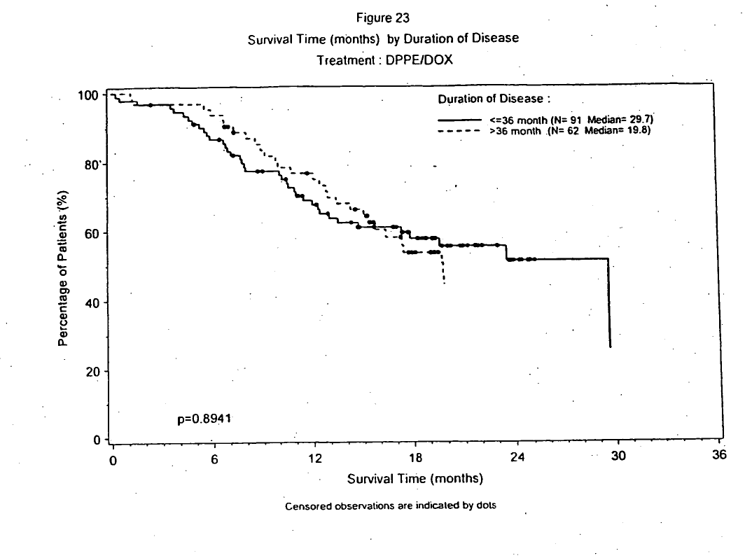

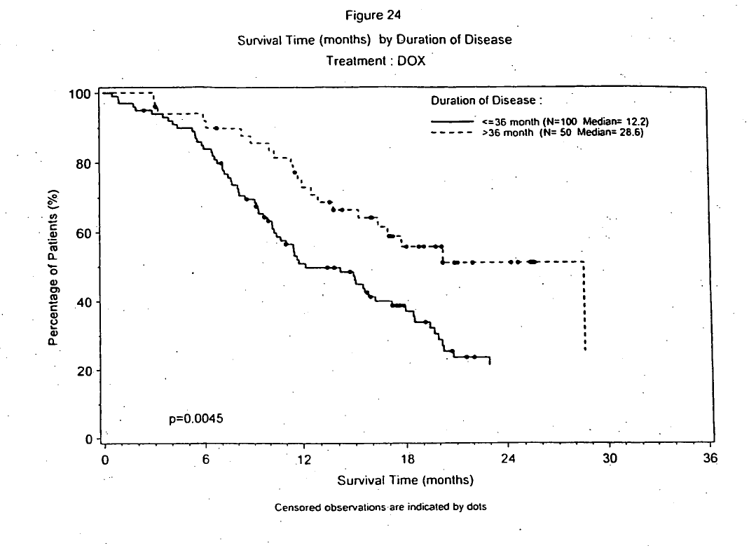

FIGS. 23 and 24 show the comparison of the survival of patients in the DPPE arm (FIG. 23) or the DOX arm (FIG. 24) when divided into subgroups (i) of less than or equal to 36 months duration and (ii) greater than 36 months duration.

These data show that, in the DOX arm, the >36 month duration patients have a more prolonged survival compared with the <36 month group. This is well known and is a consequence of the inherent indolence of the cancer in the late relapsers. In the patients receiving treatment with DPPE, those in the <36 month group behave as if they were late relapsers, i.e. they come to resemble the >36 month group in which the cancers are indolent rather than aggressive.

### Example VII

### Survival Time by Prior Treatment

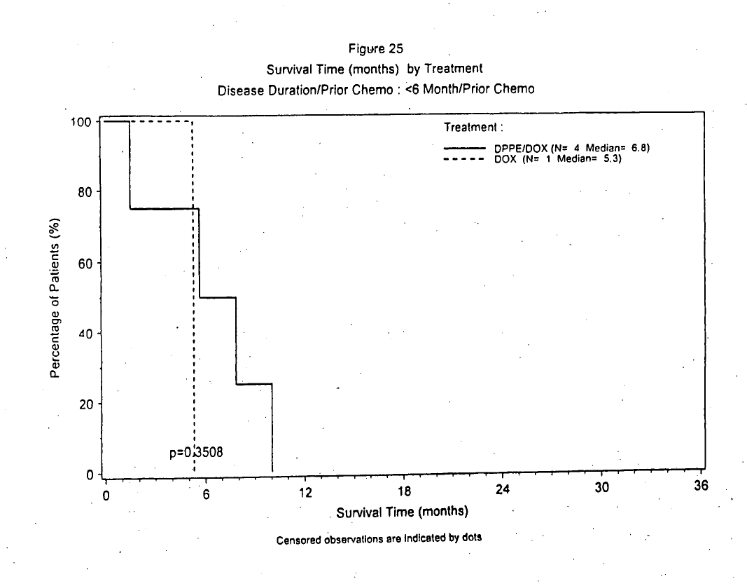

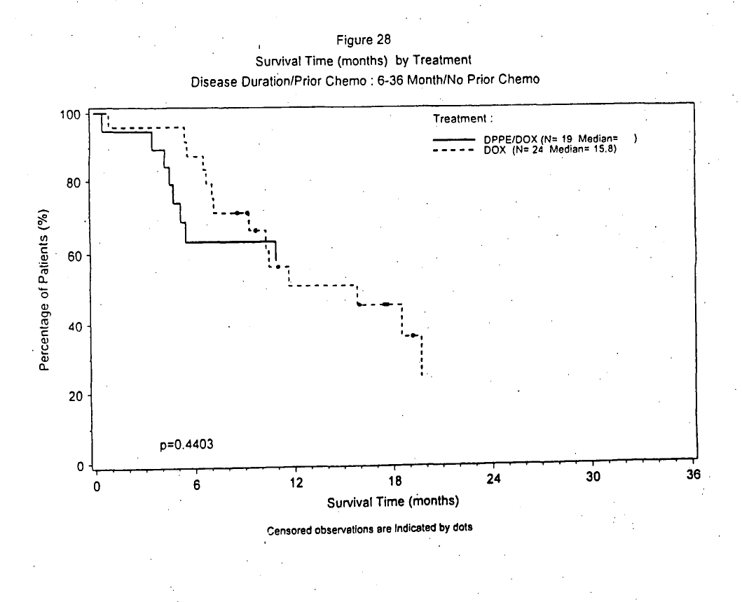

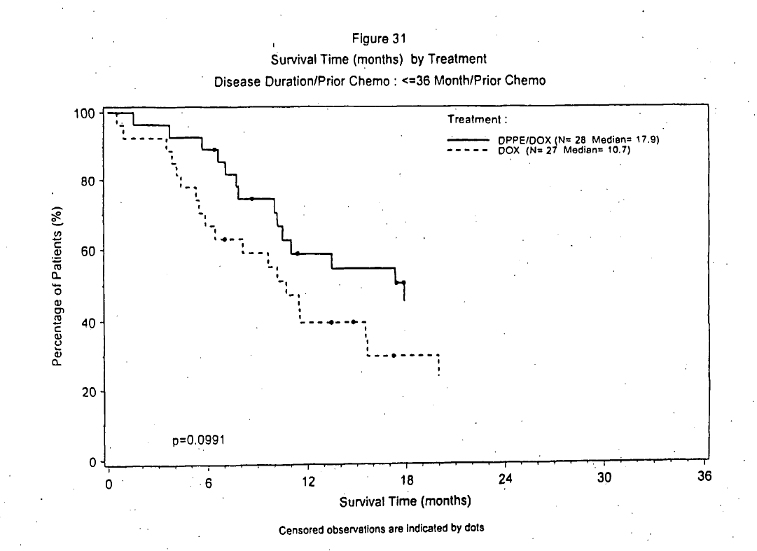

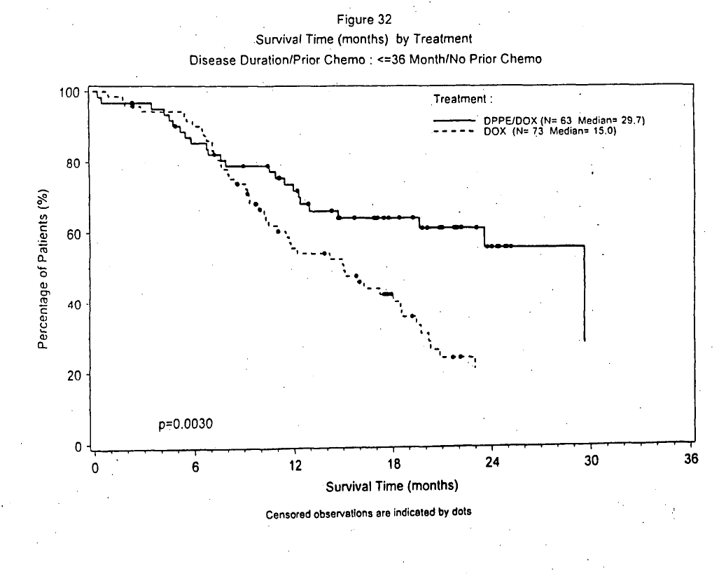

For this subgroup analysis, the 3 duration subgroups (<6 m, 6-36 m and >36 m) are further subdivided into groups of patients that (i) have undergone prior chemotherapy, and (ii) have not undergone prior chemotherapy. The overall breakdown of the number of patients in the trial who had undergone chemotherapy is provided in Table 8. The results of the analysis are shown in FIGS. 25 to 32.

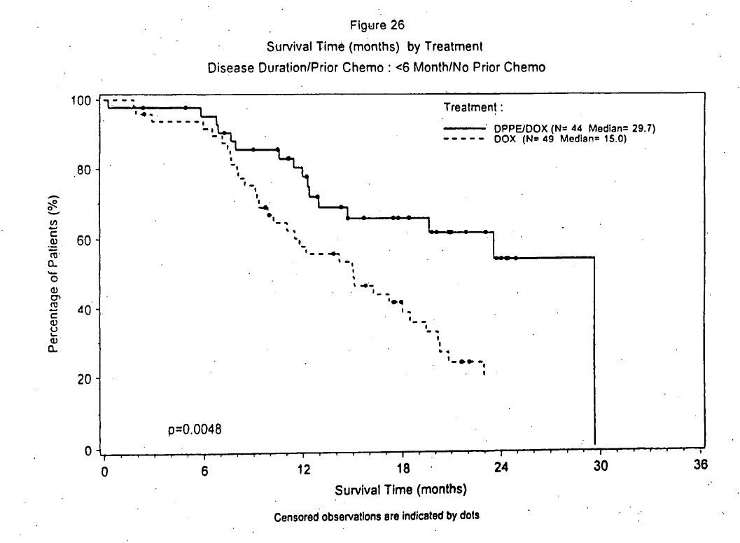

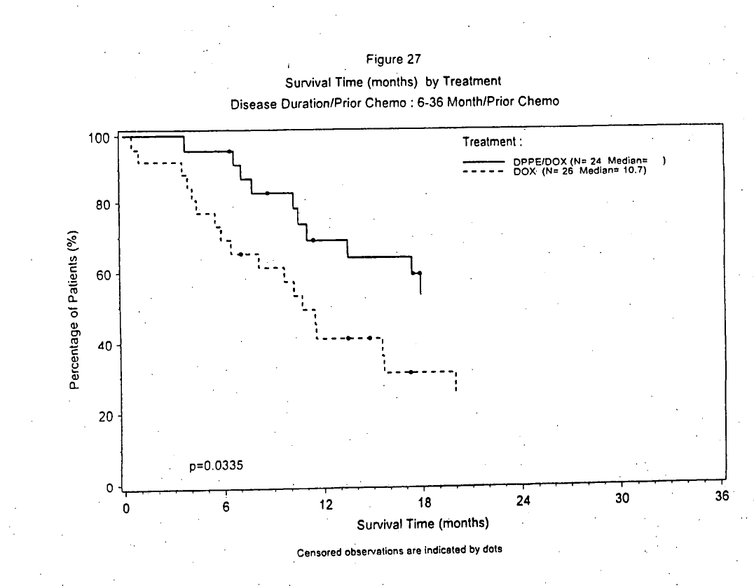

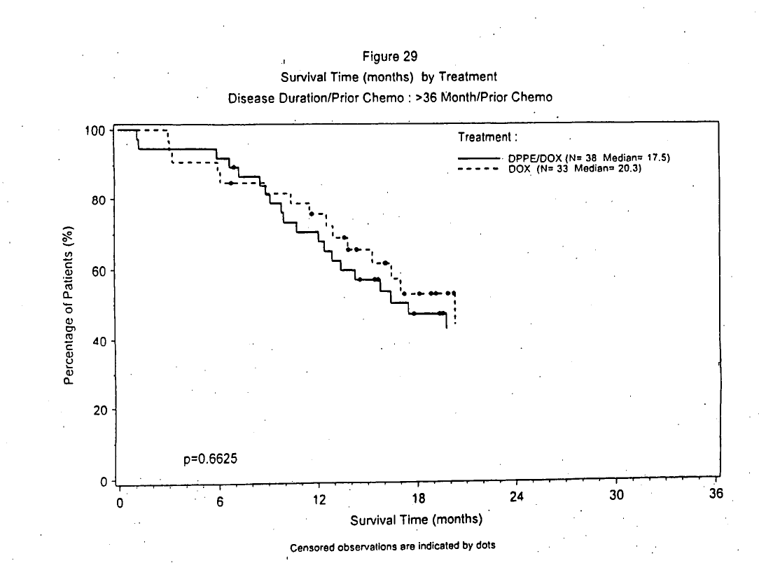

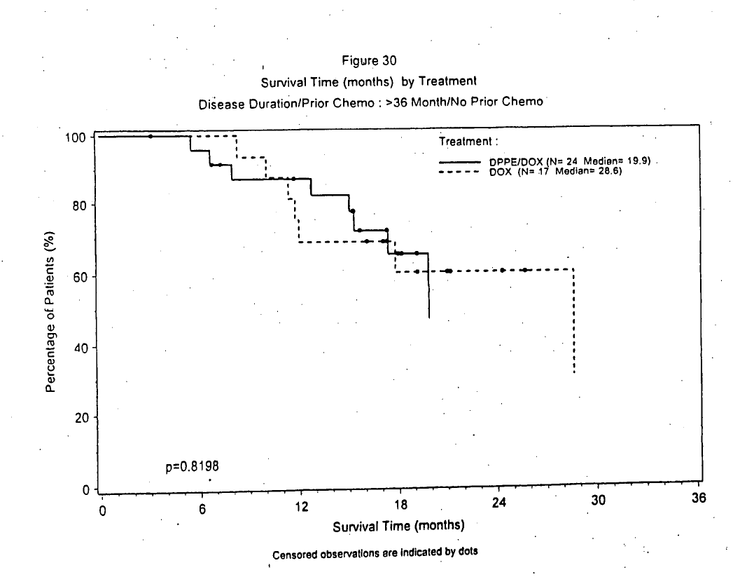

The <6 month group, which exhibited a large benefit, consists almost entirely of chemo-naïve patients and, therefore, is non-informative for this analysis (see FIGS. 25 and 26). The 6-36 month group, however, shows a greater benefit in the “prior chemotherapy group” such that the addition of DPPE extended the median survival time (MST) of this group from 10.7 months to “not yet achieved”, but >17.5 months, and the one year survival from 40% to just under 70% (p=0.0335; see FIG. 27). The “no prior chemotherapy” group (6-36 m) shows little, if any, benefit (p=0.4403). In the >36 m group, neither of the subgroups show any benefit from the addition of DPPE, as expected from the above Examples (see FIGS. 29 and 30).

These data are entirely consistent with the above conclusion that benefit from DPPE treatment is greatest in patients relapsing within 36 months, i.e. patients with aggressive disease.

The statistical finding with respect to prior chemotherapy compared to no prior chemotherapy most likely relates to the aggressivity of the tumour and the influence of adjuvant therapy on relapse date. Thus, patients with highly aggressive tumours who are not (by chance) offered adjuvant chemotherapy will relapse in <6 months. If, on the other hand, adjuvant chemotherapy is available, then these same highly aggressive tumours will relapse later, for example, between 6 and 36 months. There is, therefore, a huge benefit of DPPE treatment in the <6 months group (consisting almost exclusively of highly aggressive tumours) and also a huge benefit in the “prior chemotherapy” group (6-36 m), which also comprises highly aggressive tumours that have been delayed by 6 months or more because of the adjuvant chemotherapy (which frequently takes up to 6 months to complete).

The lack of benefit of DPPE treatment in the “no prior chemotherapy” group of 6-36 m is due to the fact that this subgroup is comprises less aggressive cancers (i.e. those that did not relapse in <6 m). The lack of benefit >36 m in either subgroup is also consistent, since both these subgroups comprise indolent cancers which had not relapsed earlier than 36 m.

### Example VIII

### Survival by ECOG Performance Status

For this analysis, the patients in the trial were divided into subgroups based on Eastern Cooperative Oncology Group (ECOG) Performance Status (PS). ECOG PS is a widely accepted standard for the assessment of the progression of a patient's disease as measured by functional impairment in patients, with ECOG PS1 indicating no functional impairment and ECOG PS 1 and 2 indicating that patients have progressively greater functional impairment but are still ambulatory.

The results of this analysis are provided in Table 9 (note the sample size in the PS 2 subgroup is too small to be meaningful). This analysis indicates that, although there were a few more PS 0 patients in the DPPE/DOX arm (69 compared to 60), the maintenance of a statistically significant difference (p=0.0459) in this subgroup excludes this minor maldistribution as being responsible for the overall survival benefit from DPPE in the whole trial. In the ECOG PS 1 subgroup, there is a trend to benefit for DPPE, the large MST difference in PS2 patients does not achieve significance because of the very small sample size.

### Example IX

### Survival by Geographic Distribution

In this example, the patients were divided into subgroups of those patients that originated from Eastern Europe and those that originated mainly from Western Europe and North America. Those patients that originated from Eastern Europe were excluded and the above analyses (Examples II-V and VIII) were conducted on the second subgroup. The results are summarised in Table 10 and compared to those for the all patients enrolled in the trial.

Similar survival rates were found for the geographically restricted subgroup when compared to the trial overall. The p values vary slightly due to the fewer numbers of patients being analysed once those in the Eastern Europe subgroup have been excluded. The majority of the parameters, including and especially survival, do not vary between the Western patients (the “Geographically restricted subset” in Table 10) and the trial as a whole. This is an important conclusion as it eliminates the possibility that the results of the trial may be influenced by the type of treatment and/or care available to the patients prior to their enrolment or after discontinuation of the DPPE treatment.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

TABLE 1*
Effect of DPPE on ER− and ER+ Patients (Only ER Known
Patients)

| | | |
|---|---|---|
| ER status | ER+ | ER− |

| (known) | n | MST¹ | 1 yr | n | MST | 1 yr |
|---|---|---|---|---|---|---|
| DOX/DPPE | 40 | 20+ m | 70% | 43 | 15 m | 65% |
| DOX | 33 | 16.5 m | 62% | 41 | 9.5 m | 36% |

| | | |
|---|---|---|
| p value | 0.1985 (NS) | 0.003 |
| Figure | 2 | 1 |

*Does not include imputed data
¹median survival time in months (m)

TABLE 2
ER values were known in 157 patients, and unknown in 146

| | Negative | Positive | Unknown |
|---|---|---|---|
| DPPE/DOX | 43 | 40 | 70 |
| DOX | 41 | 33 | 76 |

TABLE 3
Median survival time (MST) and one year survival (1 yr) by
‘duration’ data in Table 1 is reproduced below with p values
and n values inserted.

| Du- | | ≦6 m | | | >6 but <36 m | | | ≧36 m | |
|---|---|---|---|---|---|---|---|---|---|
| ration | n | MST | 1 yr | n | MST | 1 yr | n | MST | 1 yr |
| DOX/ | 48 | 23.5 m | 71% | 43 | 17.9 m+ | 65% | 62 | 19.9 m | 77% |
| DPPE | | | | | | | | | |
| DOX | 50 | 15 m | 56% | 50 | 11.5 m | 45% | 50 | 20.3 m | 77% |

| | | | |
|---|---|---|---|
| p value | 0.0192 | 0.0337 | 0.7485 (NS) |
| Fig- | 5 | 6 | 7 |
| ure | | | |

TABLE 4
Survival Information

| Re- | | CR/PR¹ | | | SD² | | | PD³ | |
|---|---|---|---|---|---|---|---|---|---|
| sponse | n | MST | 1 yr | n | MST | 1 yr | n | MST | 1 yr |
| DOX/ | 44 | 29.5 m | 87% | 67 | 19 m+ | 80% | 29 | 10 m | 38% |
| DPPE | | | | | | | | | |
| DOX | 44 | 28.5 m | 74% | 68 | 17 m | 59% | 23 | 10.5 m | 34% |

| | | | |
|---|---|---|---|
| p value | 0.1201 | 0.0075 | 0.9232 (NS) |

¹CR/PR = complete response and/or partial response
²SD = stable disease
³PD = progressive disease

TABLE 5
Distribution of ER according to duration for all patients

| | | ≦6 m | | 6-36 m | | ≧36 m | | All | |
|---|---|---|---|---|---|---|---|---|---|
| ER | Duration | n | (%) | n | % | n | % | n | % |
| Negative | | 36 | (36.7) | 34 | (36.6) | 14 | (12.5) | 84 | (27.7) |
| Positive | | 17 | (17.3) | 22 | (23.7) | 34 | (30.4) | 73 | (24.1) |
| Unknown | | 45 | (45.9) | 37 | (39.8) | 64 | (57.1) | 146 | (48.2) |
| TOTAL | | 98 | (100.0) | 93 | (100.0) | 112 | (100.0) | 303 | (100.0) |

TABLE 6
Distribution of ER according to duration for only patients with
known ER status

| | | ≦6 m | | 6-36 m | | ≧36 m | | All | |
|---|---|---|---|---|---|---|---|---|---|
| ER | Duration | n | (%) | n | % | n | % | n | % |
| Negative | | 36 | (67.9) | 34 | (60.7) | 14 | (29.2) | 84 | (53.5) |
| Positive | | 17 | (32.1) | 22 | (39.3) | 34 | (70.8) | 73 | (46.5) |
| TOTAL | | 53 | (100.0) | 56 | (100.0) | 48 | (100.0) | 157 | (100.0) |

TABLE 7
Patients with known ER status.

| | | >36 m, ER+ | | | >m, ER− | | | >36M, ER+ | | | >36 m, ER− | |
|---|---|---|---|---|---|---|---|---|---|---|---|---|
| | n | MST | 1 yr | n | MST | 1 yr | n | MST | 1 yr | N | MST | 1 yr |
| DPPE/ | 17 | 20 + m | 77% | 22 | 13.5 m | 60% | 23 | 19.5 m | 68% | 11 | 15 m | 81% |
| DOX | | | | | | | | | | | | |
| DOX | 22 | 15.5 m | 52% | 38 | 9.5 m | 36% | 11 | 18 m | 82% | 3 | 12 m | 66% |

| | | | | |
|---|---|---|---|---|
| p value | 0.0953 | 0.0034 | 0.9074 | 0.7827 |
| Figure | 8 | 9 | 10 | 11 |

TABLE 8
Prior Chemotherapy Status

| | Prior Chemotherapy | | | No Prior |
|---|---|---|---|---|
| | Adjuvant | Metastatic | Total | Chemotherapy |
| DPPE/DOX | 57 | 9 | 66 (43%) | 87 (57%) |
| DOX | 50 | 11 | 61 (41%) | 91 (59%) |

TABLE 9
Survival time by ECOG status

| ECOG | | PS 0 | | | PS 1 | | | PS 2 | |
|---|---|---|---|---|---|---|---|---|---|
| PS | n | MST | 1 yr | n | MST | 1 yr | n | MST | 1 yr |
| DOX/ | 69 | 29.5 m | 85% | 73 | 15.3 m | 63% | 11 | 10.9 m | 27% |
| DPPE | | | | | | | | | |
| DOX | 60 | 20.3 m | 75% | 78 | 11.7 m | 48% | 12 | 5.9 m | 33% |

| | | | |
|---|---|---|---|
| p value | 0.0459 | 0.1250 | 0.5974 |

TABLE 10
Analysis by Geographical Distribution and Overall Summary

| | | | Geographically restricted | | |
|---|---|---|---|---|---|
| Parameter | Whole trial | | subset | | Interpretation |
| Overall survival | MST | 23.6 m vs 15.6 m | MST | 17.5 m vs 12 m | Consistent for |
| | | p = 0.008 (unstratified) | | p = 0.0723 | this very |
| | | p = 0.021 (stratified) | | | important |
| | 1 yr | 72% vs 60% | 1 yr | 70% vs 50% | endpoint, |
| | | | | (FIG. 1) | similar |
| | | | | | proportional |
| | | | | | increase in |
| | | | | | survival in |
| | | | | | favour of DPPE. |
| Survival by | MST | 18 + m vs 11.5 m | MST | 17 m vs 9 m | Consistent and |
| duration: 6- | | p = 0.0337 | | p = 0.18 | similar |
| 36 m | | | | | |
| <6 m | | | | N/A | |
| >36 m | | | | N/A | |
| Survival by ER | MST | 15 m vs 9.5 m | MST | 15 m vs 9 m | Very |
| status ER- | 1 yr | 65% vs 36% | 1 yr | 68% vs 39% | consistently in |
| | | p = 0.003 | | p = 0.0674 | favour of DPPE |
| Survival by | MST | 20 + m vs 16 m | MST | 19 m vs 15.5 m | Very similar |
| ER+ | 1 yr | 70% vs 62% | 1 yr | 65% vs 55% | graphs |
| | | p = 0.1985 | | p = 0.3821 | |
| DPPE/DOX for | ER+ slightly better than ER− | | ER+ slightly better than ER− | | Very similar |
| ER+ vs ER− | | p = 0.1121 | | p = 0.2510 | graphs |
| DOX for ER+ | ER+ considerably better than ER− | | ER+ considerably better than ER− | | Very similar |
| vs ER− | | p = 0.0021 | | p = 0.0687 | graphs |
| Survival by | 1 yr | 86% vs 74% | 1 yr | 88% vs 62% | Very similar |
| response: | | p = 0.1201 | | p = 0.2650 | graphs in favour |
| CR/PR | | | | | of DPPE |
| Survival by | MST | 19 + m vs 17 m | MST | 17.5 m vs 12 m | Bigger |
| response: SD | 1 yr | 80% vs 59% | 1 yr | 73% vs 53% | difference in |
| | | p = 0.0075 | | p = 0.2650 | favour of DPPE |
| | | | | | for whole trial |
| Survival by | Superimposable | | DPPE a little better at first | | Fairly similar |
| response: PD | | p = 0.9232 | | p = 0.7664 | |
| Survival by | MST | 29.5 m vs 20.3 m | MST | 29.5 m vs 16.5 m | Looks very |
| ECOG = 0 | 1 yr | 85% vs 75% | 1 yr | 85% vs 63% | similar |
| | | p = 0.0459 | | p not given | |
| Survival by | MST | 15.2 m vs 11.7 m | MST | 13.5 m vs 11.5 m | Looks similar |
| ECOG = 1 | 1 yr | 63% vs 48% | 1 yr | 59% vs 42% | |
| | | p = 0.1250 | | p not given | |

1-31. (Cancelled)

32. A method of treating a cancer patient having, or suspected of having, an aggressive breast cancer, which comprises administering N,N-diethyl-2-[-4-(phenylmethyl)-phenoxy]ethanamine monohydrochloride (DPPE) to the patient to thereby extend the survival of said patient.

33. The method of claim 32 wherein said aggressive cancer is a breast cancer or has metastasized from a breast cancer.

34. The method of claim 33 wherein the aggressive cancer is such in that the patient requires chemotherapy for advanced disease in 40 months or less following a diagnosis of a primary cancer.

35. The method of claim 34 wherein the patient requires chemotherapy in 36 months or less following diagnosis.

36. The method of claim 33 wherein the breast cancer is estrogen receptor negative.

37. The method of claim 34 wherein the primary cancer is estrogen receptor negative.

38. The method of claim 34 wherein the primary cancer is estrogen receptor positive.

39. The method of claim 35 wherein the breast cancer is a primary cancer which is estrogen receptor negative.

40. The method of claim 32 wherein DPPE is administered in combination with a second chemotherapeutic agent.

41. The method of claim 32 wherein DPPE is administered concurrently with DPPE.

42. The method of claim 33 wherein DPPE is administered concurrently with DPPE.

43. The method of claim 35 wherein DPPE is administered concurrently with DPPE.

44. The method of claim 36 wherein DPPE is administered concurrently with DPPE.

45. The method of claim 43 wherein said second chemotherapeutic agent is an anthracycline or a taxane.

46. The method of claim 43 wherein said second chemotherapeutic agent is doxorubicin.

47. The method of claim 32 which is an adjuvant therapy to treat said patient.

48. The method of claim 47 wherein said suspected cancer is a breast cancer or has metastasized from a breast cancer which is estrogen receptor negative.

49. The method of claim 32 which is effected following surgery for treatment of a primary cancer which is an estrogen negative cancer.

50. The method of claim 49 wherein the DPPE is administered in combination with a second chemotherapeutic agent.

51. The method of claim 49 wherein DPPE is administered concurrently with a second chemotherapeutic agent.

52. The method of claim 51 wherein said second chemotherapeutic agent is an anthracycline or a taxane.

53. The method of claim 52 wherein said second chemotherapeutic agent is doxorubicin.

54. The method of claim 44 wherein said second chemotherapeutic agent is an anthracycline or a taxane.

55. The method of claim 44 wherein said second chemotherapeutic agent is doxorubicin.
